SEQUENCE LISTING (2 sequences)

SEQ ID NO: 1 - RNA, 47 nt, Artificial
  RNA template
gggucucucugguuagaccacucuagcaguggcgcccgaacagggac

SEQ ID NO: 2 - DNA, 18 bp, artificial
  5′ g is a biotinylated guanosine
gtccctgttcgggcgcca

(19) United States
(12) Patent Application Publication
(10) Pub. No.: US 2008/0293664 A1
(43) Pub. Date: Nov. 27, 2008

(54) Non-nucleoside reverse transcriptase inhibitors

(75) Inventors: Roland Joseph Billedeau, Santa Clara, CA (US); Wylie Solang Palmer, Mountain View, CA (US); Zachary Kevin Sweeney, Redwood City, CA (US); Jeffrey Wu, Saratoga, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 12/082,069

(22) Filed: Apr. 8, 2008

Related U.S. Application Data
(60) Provisional application No. 60/922,449, filed on Apr. 9, 2007.
(60) Provisional application No. 60/961,346, filed on Jul. 20, 2007.

(51) Int. Cl. A61K 31/5025; A61P 31/18; C07D 471/04; A61K 31/7072; A61K 31/7068; A61K 31/551; A61K 31/513; A61K 31/496; A61K 31/538; A61K 31/708; A61K 31/437; C07D 487/04

(52) U.S. Cl. 514/45; 546/119; 544/236; 514/248; 514/303; 514/50; 514/49; 514/230.5; 514/220; 514/253.09; 514/274

(57) ABSTRACT

Compounds of formula I, wherein R¹, R², R³, R⁴, R^{a}, X, X¹, and Y are as defined herein or pharmaceutically acceptable salts thereof, inhibit HIV-1 reverse transcriptase and afford a method for prevention and treatment of HIV-1 infections and the treatment of AIDS and/or ARC. The present invention also relates to compositions containing compounds of formula I useful for the prevention and treatment of HIV-1 infections and the treatment of AIDS and/or ARC.

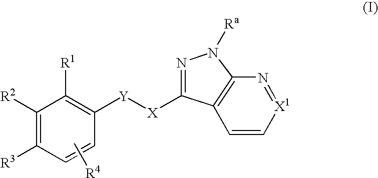

### CROSS REFERENCE TO PRIOR APPLICATIONS

This application is claims benefit of U.S. Provisional Application No. 60/922,449, filed Apr. 9, 2007 and U.S. Provisional Application No. 60/961,346, filed Jul. 20, 2007 both of which are hereby incorporated by reference in its entirety.

### FIELD OF THE INVENTION

The invention relates to the field of antiviral therapy and, in particular, to non-nucleoside compounds that inhibit HIV-1 reverse transcriptase and are useful for treating Human Immunodeficiency Virus (HIV) mediated diseases. The invention provides novel 1H-pyrazolo[3,4-c]pyridazinyl, 1H-pyrazolo[3,4-b]pyridinyl, 1H-pyrazolo[3,4-c]pyridinyl and indazolyl compounds, pharmaceutical compositions comprising these compounds, methods for treatment or prophylaxis of HIV-1 mediated diseases employing said compounds in monotherapy or in combination therapy.

### BACKGROUND OF THE INVENTION

The human immunodeficiency virus HIV is the causative agent of acquired immunodeficiency syndrome (AIDS), a disease characterized by the destruction of the immune system, particularly of the CD4+ T-cell, with attendant susceptibility to opportunistic infections. HIV infection is also associated with a precursor AIDS-related complex (ARC), a syndrome characterized by symptoms such as persistent generalized lymphadenopathy, fever and weight loss.

In common with other retroviruses, the HIV genome encodes protein precursors known as gag and gag-pol which are processed by the viral protease to afford the protease, reverse transcriptase (RT), endonuclease/integrase and mature structural proteins of the virus core. Interruption of this processing prevents the production of normally infectious virus. Considerable efforts have been directed towards the control of HIV by inhibition of virally encoded enzymes.

Currently available chemotherapy targets two crucial viral enzymes: HIV protease and HIV reverse transcriptase. (J. S. G. Montaner et al., Antiretroviral therapy: ‘the state of the art’, Biomed. & Pharmacother. 1999 53:63-72; R. W. Shafer and D. A. Vuitton, Highly active retroviral therapy (HAART) for the treatment of infection with human immunodeficiency virus type, Biomed. & Pharmacother. 1999 53 :73-86; E. De Clercq, New Developments in Anti-HIV Chemotherap. Curr. Med. Chem. 2001 8:1543-1572). Two general classes of RTI inhibitors have been identified: nucleoside reverse transcriptase inhibitors (NRTI) and non-nucleoside reverse transcriptase inhibitors. Currently the CCR5 co-receptor has emerged as a potential target for anti-HIV chemotherapy (D. Chantry, Expert Opin. Emerg. Drugs 2004 9(1):1-7; C. G. Barber, Curr. Opin. Invest. Drugs 2004 5(8):851-861; D. Schols, Curr. Topics Med. Chem. 2004 4(9):883-893; N. A. Meanwell and J. F. Kadow, Curr. Opin. Drug Discov. Dev. 2003 6(4):451-461). Drugs targeted at new enzymatic targets have entered the market including integrase inhibitors typified by Raltegravir (Merck) has been approved by the FDA and Elvitegravir (Gilead Sciences and Japan Tobacco) is in phase II trials. The CCR5 antagonist maraviroc (SELZENTRY™, Pfizer) has also been approved by the FDA for anti-HIV-1 therapy.

NRTIs typically are 2′,3′-dideoxynucleoside (ddN) analogs which must be phosphorylated prior to interacting with viral RT. The corresponding triphosphates function as competitive inhibitors or alternative substrates for viral RT. After incorporation into nucleic acids the nucleoside analogs terminate the chain elongation process. HIV reverse transcriptase has DNA editing capabilities which enable resistant strains to overcome the blockade by cleaving the nucleoside analog and continuing the elongation. Currently clinically used NRTIs include zidovudine (AZT), didanosine (ddI), zalcitabine (ddC), stavudine (d4T), lamivudine (3TC) and tenofovir (PMPA).

NNRTIs were first discovered in 1989. NNRTIs are allosteric inhibitors which bind reversibly at a nonsubstrate-binding site on the HIV reverse transcriptase thereby altering the shape of the active site or blocking polymerase activity (R. W. Buckheit, Jr., Non-nucleoside reverse transcriptase inhibitors: perspectives for novel therapeutic compounds and strategies for treatment of HIV infection, Expert Opin. Investig. Drugs 2001 10(8)1423-1442; E. De Clercq, The role of non-nucleoside reverse transcriptase inhibitors (NNRTIs) in the therapy of HIV infection, Antiviral Res. 1998 38:153-179; E. De Clercq, New Developments in Anti-HIV Chemotherapy, Current medicinal Chem. 2001 8(13): 1543-1572; G. Moyle, The Emerging Roles of Non-Nucleoside Reverse Transcriptase Inhibitors in Antiviral Therapy, Drugs 2001 61 (1): 19-26). Although over thirty structural classes of NNRTIs have been identified in the laboratory, only four compounds have been approved for HIV therapy: efavirenz, nevirapine, delavirdine and etravirine.

Initially viewed as a promising class of compounds, in vitro and in vivo studies quickly revealed the NNRTIs presented a low barrier to the emergence of drug resistant HIV strains and class-specific toxicity. Drug resistance frequently develops with only a single point mutation in the RT. While combination therapy with NRTIs, PIs and NNRTIs has, in many cases, dramatically lowered viral loads and slowed disease progression, significant therapeutic problems remain. (R. M. Gulick, Eur. Soc. Clin. Microbiol. and Inf. Dis. 2003 9(3):186-193) The cocktails are not effective in all patients, potentially severe adverse reactions often occur and the rapidly reproducing HIV virus has proven adroit at creating mutant drug-resistant variants of wild type protease and reverse transcriptase. There remains a need for safer drugs with activity against wild type and commonly occurring resistant strains of HIV.

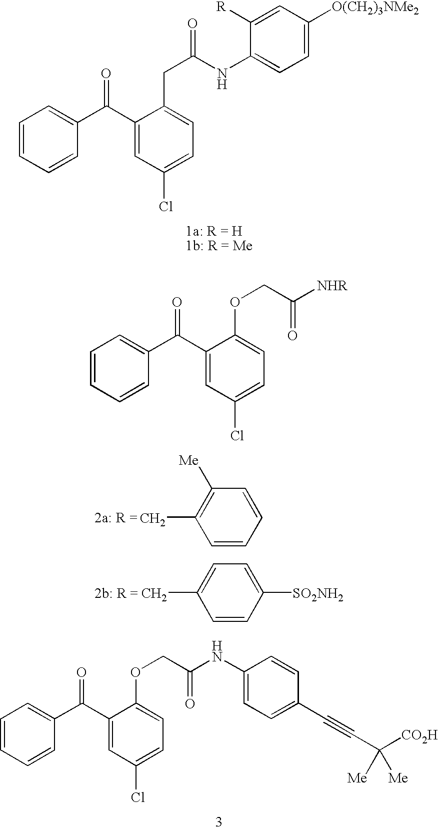

2-Benzoyl phenyl-N-[phenyl]-acetamide compounds 1a and 1b have been shown to inhibit HIV-1 reverse transcriptase (P. G. Wyatt et al., J. Med. Chem. 1995 38(10):1657-1665). Further screening identified related compounds, e.g. 2-benzoyl phenyloxy-N-[phenyl]-acetamide, 2a, and a sulfonamide derivative 2b which also inhibited reverse transcriptase (J. H. Chan et al., J. Med Chem. 2004 47(5):1175-1182; K. Romimes et al., J. Med. Chem. 2006 49(2):727-739; C. L. Webster et al., WO01/1 7982). P. Bonneau et al. in US 20060069261 published Mar. 30, 2006 disclose 4-{4-[2-(2-benzoyl-phenoxy)-acetylamino]-phenyl}-2,2-dimethyl-but-3-ynoic acid compounds 3 which are inhibitors of HIV reverse transcriptase.

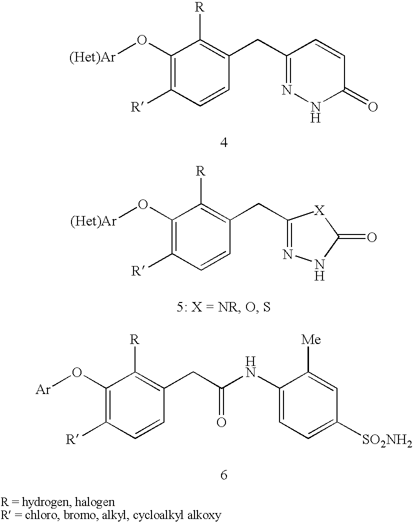

Pyridazinone non-nucleoside reverse transcriptase inhibitors 4 have been described by J. P. Dunn et al. in U.S. Patent Publication 20040198736 filed Mar. 23, 2004 and by J. P. Dunn et al. in U.S. Publication No. 2005021554 filed Mar. 22, 2005. 5-Aralkyl-2,4-dihydro-[1,2,4]triazol-3-one, 5-aralkyl-3H-[1,3,4]oxadiazol-2-one and 5-aralkyl-3H-[1,3,4]thiadiazol-2-one non-nucleoside reverse transcriptase inhibitors 5 have been disclosed by J. P. Dunn et al. in U.S. Publication No. 20040192704 filed Mar. 23, 2004 and by J. P. Dunn et al. in U.S. Publication No. 20060025462 filed Jun. 27, 2005. Related compounds are disclosed by Y. D. Saito et al. in U.S. Pub. No. 20070078128 filed Sep. 29, 2006. Phenylacetamide non-nucleoside reverse transcriptase inhibitors 6 have been disclosed by J. P. Dunn et al. in U.S. Pub. No. 20050239881 published Oct. 27, 2005 and methods for treating retroviral infection with phenylacetamide compounds have been disclosed by J. P. Dunn et al. in U.S. Publication No. 20050239880 published Oct. 27, 2005; T. Mirzadegan and T. Silva in U.S. Publication No. 20070088015 filed Oct. 18, 2006; and Z. K. Sweeney and T. Silva in U.S. Publication No 20070088053 Oct. 18, 2006. These applications are hereby incorporated by reference in their entirety.

Novel 1H-pyrazolo[3,4-c]pyridazinyl, 1H-pyrazolo[3,4-b]pyridinyl, 1H-pyrazolo[3,4-c]pyridinyl and indazolyl compounds, pharmaceutical compositions comprising these compounds and methods for treatment or prophylaxis of HIV-1 mediated diseases employing said compounds in monotherapy or in combination therapy were disclosed by J. Kennedy-Smith et al. in U.S. Patent Publication 20080045511, published Feb. 21, 2008 which is hereby incorporated by reference in its entirety.

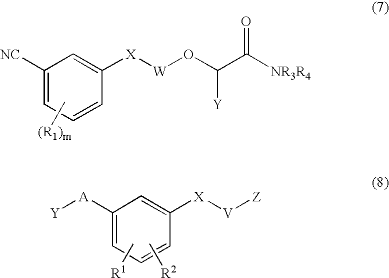

In WO2006/067587 published Jun. 26, 2006, L. H. Jones et al. disclose biaryl ether derivatives 7 and compositions containing them which bind to the enzyme reverse transcriptase and are modulators, especially inhibitors, thereof. In U.S. Patent Publication 2007/0021442 published Jan. 25, 2007, S. A. Saggar et al. disclose HIV reverse transcriptase inhibitors of formula 8.

### SUMMARY OF THE INVENTION

The present invention relates to a compound according to formula I

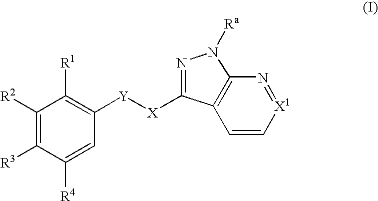

wherein:

X is CH₂ or NH;

Y is CH₂ or O with the proviso that at least one of X or Y is CH₂; and with the further proviso that when X¹ is CH, either (i) R¹ is OAr or C(═O)Ar or (ii) X is NH

X¹ is N or CH;

R¹ is C(═O)Ar, OAr, fluorine or hydrogen;

R² is OAr, hydrogen, halogen, C₁₋₆ alkyl, C₁₋₆ alkoxy or C₃₋₅ cycloalkyl;

R³ and R⁴ are independently hydrogen, halogen, C₁₋₆ alkyl, C₁₋₆ alkoxy or C₃₋₅ cycloalkyl;

R^{a} is hydrogen, CH₂OH, CH₂C(═O)(CH₂)ₙC(═O)OH where n is 2 to 5, CH₂C(═O)C₁₋₆ alkyl, or CH₂C(═O)CHR^{b}NH₂ where R^{b} is phenyl or C₁₋₆ lower alkyl;

Ar is phenyl substituted with 1 to 3 groups independently selected from halogen, cyano, C₁₋₆ haloalkyl or C₁₋₆ alkyl; or,

pharmaceutically acceptable salts thereof.

Compounds of formula I inhibit HIV-1 reverse transcriptase and afford a method for prevention and treatment of HIV-1 infections and the treatment of AIDS and/or ARC. HIV-1 undergoes facile mutations of its genetic code resulting in strains with reduced susceptibility to therapy with current therapeutic options. The present invention also relates to compositions containing compounds of formula I useful for the prevention and treatment of HIV-1 infections and the treatment of AIDS and/or ARC. The present invention further relates to compounds of formula I which are useful in mono therapy or combination therapy with other anti-viral agents.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

A “stable” compound is a compound which can be prepared and isolated and whose structure and properties remain or can be made to remain essentially unchanged for a period of time sufficient to allow the use of the compound for the purposes described herein (e.g., therapeutic or prophylactic administration to a subject).

Unless expressly stated to the contrary, all ranges cited herein are inclusive. For example, a heterocyclic ring described as containing “1 to 4 heteroatoms” means the ring can contain 1, 2, 3 or 4 heteroatoms. It is also to be understood that any range cited herein includes within its scope all of the subranges within that range. Thus, for example, an aryl or a heteroaryl described as optionally substituted with “from 1 to 5 substituents” is intended to include as aspects thereof, any aryl optionally substituted with 1 to 4 substituents, 1 to 3 substituents, 1 to 2 substituents, 2 to 5 substituents, 2 to 4 substituents, 2 to 3 substituents, 3 to 5 substituents, 3 to 4 substituents, 4 to 5 substituents, 1 substituent, 2 substituents, 3 substituents, 4 substituents, and 5 substituents]

The symbols “*” at the end of a bond or

[symbol]

drawn through a bond each refer to the point of attachment of a functional group or other chemical moiety to the rest of the molecule of which it is a part. Thus, for example:

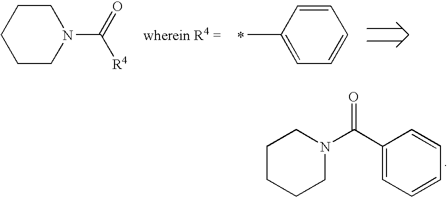

It is contemplated that the definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, 3 phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as 6 used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

In one embodiment of the present invention there is provided a compound according to formula I wherein R¹, R², R³, R⁴, R^{a}, R^{b}, Ar, X, X¹, Y and n are as defined herein above.

In a second embodiment of the present invention there is provided a compound according to formula wherein R¹ is hydrogen or fluorine and R² is OAr.

In a third embodiment of the present invention there is provided a compound according to formula I wherein R¹ is fluoro; R² is OAr; R³ is halogen, C₁₋₆ alkyl, C₁₋₆ alkoxy or C₃₋₅ cycloalkyl; and R⁴ and R^{a} are hydrogen.

In another embodiment of the present invention there is provided a compound according to formula I wherein R¹ is fluoro; R² is OAr; R³ is halogen, C₁₋₆ alkyl, C₁₋₆ alkoxy or C₃₋₅ cycloalkyl; R⁴ is hydrogen and R^{a} is CH₂C(═O)(CH₂)ₙC(═O)OH where n is 2 to 5.

In a fourth embodiment of the present invention there is provided a compound according to formula I wherein R¹ is fluoro; R² is OAr; R³ is halogen, C₁₋₆ alkyl, C₁₋₆ alkoxy or C₃₋₅ cycloalkyl; R⁴ and R^{a} are hydrogen; and Ar is 3,5-disubstituted phenyl wherein one substituent is cyano and the other substituent is halogen, cyano or C₁₋₆ haloalkyl.

In a fifth embodiment of the present invention there is provided a compound according to formula I wherein R¹ is fluoro; R² is OAr; R³ is halogen, C₁₋₆ alkyl, C₁₋₆ alkoxy or C₃₋₅ cycloalkyl; R⁴ and R^{a} are hydrogen; Ar is 3,5-disubstituted phenyl wherein one substituent is cyano and the other substituent is halogen, cyano or C₁₋₄ haloalkyl; X¹ is N; X is CH₂; and Y is CH₂ or O.

In a sixth embodiment of the present invention there is provided a compound according to formula I wherein R¹ is fluoro; R² is OAr; R³ is halogen, C₁₋₆ alkyl, C₁₋₆ alkoxy or C₃₋₅ cycloalkyl; R⁴ and R^{a} are hydrogen; Ar is 3,5-disubstituted phenyl wherein one substituent is cyano and the other substituent is halogen, cyano or C₁₋₆ haloalkyl; X¹ is N; X is CH₂; and Y is O.

In a seventh embodiment of the present invention there is provided a compound according to formula I wherein R¹ is fluoro; R² is OAr; R³ is halogen, C₁₋₆ alkyl, C₁₋₆ alkoxy or C₃₋₅ cycloalkyl; R^{a} is hydrogen; Ar is 3,5-disubstituted phenyl wherein one substituent is cyano and the other substituent is halogen, cyano or C₁₋₆ haloalkyl; X¹ is N; X is CH₂; and Y is CH₂.

In an eighth embodiment of the present invention there is provided a compound according to formula I wherein R¹ is fluoro; R² is OAr; R³ is halogen, C₁₋₆ alkyl, C₁₋₆ alkoxy or C₃₋₅ cycloalkyl; R⁴ and R^{a} are hydrogen; Ar is 3,5-disubstituted phenyl wherein one substituent is cyano and the other substituent is halogen, cyano or C₁₋₆ haloalkyl; X is NH; and Y is CH₂.

In a ninth embodiment of the present invention there is provided a compound according to formula I wherein R¹ is fluoro; R² is OAr; R³ is halogen, C₁₋₆ alkyl, C₁₋₆ alkoxy or C₃₋₅ cycloalkyl; R⁴ and R^{a} are hydrogen; Ar is 3,5-disubstituted phenyl wherein one substituent is cyano and the other substituent is halogen, cyano or C₁₋₆ haloalkyl; X is NH; and X¹ is CH.

In a tenth embodiment of the present invention there is provided a compound according to formula I wherein X¹ is N; X is CH₂; Y is CH₂ or O; R¹ is fluoro; R² is OAr; R³ is halogen, C₁₋₆ alkyl, C₁₋₆ alkoxy or C₃₋₅ cycloalkyl; R⁴ is hydrogen; Ar is a 3,5-disubstituted-phenyl wherein one substituent is cyano and the other substitutent is halogen, cyano or C₁₋₆ haloalkyl; and, R^{a} is CH₂C(═O)(CH₂)ₙC(═O)OH where n is 2 to 5.

In a eleventh embodiment of the present invention there is provided a compound according to formula I wherein R¹ and R⁴ are fluoro; R² is OAr; R³ is halogen, C₁₋₆ alkyl, C₁₋₆ alkoxy or C₃₋₅ cycloalkyl; and, R^{a} is hydrogen or CH₂C(═O)(CH₂)ₙC(═O)OH where n is 2 to 5.

In a twelfth embodiment of the present invention there is provided a compound according to formula I wherein X¹ is N; X is CH₂; Y is CH₂ or O; R¹ and R⁴ are fluoro; R² is OAr; R³ is halogen, C₁₋₄ alkyl, C₁₋₄ alkoxy or C₃₋₅ cycloalkyl; and, R^{a} is hydrogen or CH₂C(═O)(CH₂)ₙC(═O)OH where n is 2 to 5; Ar is a 3,5-disubstituted-phenyl wherein one substituent is cyano and the other substitutent is halogen, cyano or C₁₋₆ haloalkyl.

In a thirteenth embodiment of the present invention there is provided a compound according to formula IIa embodiment of the present invention there is provided a compound according to formula I wherein X¹ is N; X is CH₂; Y is CH₂ or O; R¹ and R⁴ are fluoro; R² is OAr; R³ is halogen, C₁₋₆ alkyl, C₁₋₆ alkoxy or C₃₋₅ cycloalkyl; and, R^{a} is hydrogen; Ar is a 3,5-disubstituted-phenyl wherein one substituent is cyano and the other substitutent is halogen, cyano or C₁₋₆ haloalkyl.

In a fourteenth embodiment of the present invention there is provided a compound according to 9 formula I wherein R¹ is OAr and R², R³ and R⁴ are independently hydrogen, halogen or C₁₋₆ alkyl.

In a fifteenth embodiment of the present invention there is provided a compound according to formula I wherein R¹ is OAr; Ar is 3,5-disubstituted phenyl wherein one substituent is cyano and the other substituent is halogen, cyano or C₁₋₆ haloalkyl; R⁴ is hydrogen and R^{a} is CH₂OC(═O)(CH₂)ₙC(═O)OH wherein n is 2 to 5 or hydrogen; and R² and R³ are independently hydrogen, halogen or C₁₋₆ alkyl.

In a sixteenth embodiment of the present invention there is provided a compound according to formula I wherein R¹ is OAr; Ar is 3,5-disubstituted phenyl wherein one substituent is cyano and the other substituent is halogen, cyano or C₁₋₆ haloalkyl; R⁴ and R^{a} are hydrogen; and R² and R³ are independently hydrogen, halogen or C₁₋₆ alkyl.

In yet another embodiment of the present invention there is provided a compound according to formula I wherein R¹ is OAr; Ar is 3,5-disubstituted phenyl wherein one substituent is cyano and the other substituent is halogen, cyano or C₁₋₆ haloalkyl; R⁴ is hydrogen; R^{a} is CH₂OC(═O)(CH₂)ₙC(═O)OH where n is 2 to 5; and R², R³ and R⁴ are independently hydrogen, halogen or C₁₋₆ alkyl.

In a seventeenth embodiment of the present invention there is provided a compound according to formula I wherein R¹ is OAr; Ar is 3,5-disubstituted phenyl wherein one substituent is cyano and the other substituent is halogen, cyano or C₁₋₆ haloalkyl; R⁴ is hydrogen and R^{a} is CH₂C(═O)(CH₂)ₙC(═O)OH wherein n is 2 to 5 or hydrogen; X¹ is N; and R² and R³ are independently hydrogen, halogen or C₁₋₆ alkyl.

In an eighteenth embodiment of the present invention there is provided a compound according to formula I wherein R¹ is OAr; Ar is 3,5-disubstituted phenyl wherein one substituent is cyano and the other substituent is halogen, cyano or C₁₋₆ haloalkyl; R⁴ is hydrogen and R^{a} is CH₂C(═O)(CH₂)ₙC(═O)OH wherein n is 2 to 5 or hydrogen; X¹ is CH; and R² and R³ are independently hydrogen, halogen or C₁₋₆ alkyl.

In a nineteenth embodiment of the present invention there is provided a compound according to formula I wherein R¹ is C(═O)Ar; and R² and R³ are independently hydrogen, halogen or C₁₋₆ alkyl.

In a twentieth embodiment of the present invention there is provided a compound according to formula I wherein R¹ is C(═O)Ar; Ar is 3,5-disubstituted phenyl wherein one substituent is cyano and the other substituent is halogen, cyano or C₁₋₆ haloalkyl; R^{a} is hydrogen; and R² and R³ are independently hydrogen, halogen or C₁₋₆ alkyl.

In a twenty-first embodiment of the present invention there is provided a compound according to formula I wherein R¹ is C(═O)Ar; Ar is 3,5-disubstituted phenyl wherein one substituent is cyano and the other substituent is halogen, cyano or C₁₋₆ haloalkyl; R^{a} is hydrogen; R² is halogen; and R³ is halogen or C₁₋₆ alkyl.

In a twenty-second embodiment of the present invention there is provided a compound according to formula I wherein R¹ is C(═O)Ar; Ar is 3,5-disubstituted phenyl wherein one substituent is cyano and the other substituent is halogen, cyano or C₁₋₆ haloalkyl; R² is halogen; R³ is halogen or C₁₋₆ alkyl; R^{a} is hydrogen; and X¹ is N.

In a twenty-third embodiment of the present invention there is provided a compound according to formula I wherein R¹ is C(═O)Ar; Ar is 3,5-disubstituted phenyl wherein one substituent is cyano and the other substituent is halogen, cyano or C₁₋₆ haloalkyl; R² is halogen; R³ is halogen or C₁₋₆ alkyl; R^{a} is hydrogen; and X¹ is CH.

In a twenty-fourth embodiment of the present invention there is provided a method for treating an HIV-1 infection, or preventing an HIV-1 infection, or treating AIDS or ARC, comprising administering to a host in need thereof a therapeutically effective amount of a compound according to formula I wherein R¹, R², R³, R⁴, R^{a}, R^{b}, Ar, X, X¹, Y and n are as defined herein above

In a twenty-fifth embodiment of the present invention there is provided a method for treating an HIV-1 infection, or preventing an HIV-1 infection, or treating AIDS or ARC, comprising co-administering to a host in need thereof a therapeutically effective amount of a compound according to formula I wherein R¹, R², R³, R⁴, R^{a}, R^{b}, Ar, X, X¹, Y and n are as defined herein above and a therapeutically effective amount of at least one compound selected from the group consisting of comprising co-administering at least one compound selected from the group consisting of HIV protease inhibitors, nucleoside reverse transcriptase inhibitors, non-nucleoside reverse transcriptase inhibitors, integrase inhibitors, CCR5 antagonists and viral fusion inhibitors.//In another embodiment of the present invention there is provided a method for treating an HIV-1 infection, or preventing an HIV-1 infection, or treating AIDS or ARC, comprising co-administering to a host in need thereof a therapeutically effective amount of a compound according to formula I wherein R¹, R², R³, R⁴, R^{a}, R^{b}, Ar, X, X¹, Y and n are as defined herein above and a therapeutically effective amount of at least one compound selected from the group consisting of HIV protease inhibitors, nucleoside reverse transcriptase inhibitors, non-nucleoside reverse transcriptase inhibitors, CCR5 antagonists and viral fusion inhibitors.

In a twenty-sixth embodiment of the present invention there is provided a method for treating an HIV-1 infection, or preventing an HIV-1 infection, or treating AIDS or ARC, comprising co-administering to a host in need thereof a therapeutically effective amount of a compound according to formula I wherein R¹, R², R³, R⁴, R⁵, R⁶, R^{a}, R^{b}, Ar, X, X¹, Y and n are as defined herein above and a therapeutically effective amount of at least one compound selected from the group consisting of zidovudine, lamivudine, didanosine, zalcitabine, stavudine, rescriptor, sustiva, viramune, efavirenz, etravirine, nevirapine, delavirdine, saquinavir, ritonavir, nelfinavir, indinavir, amprenavir, lopinavir, enfuvirtide, maraviroc or raltegravir. In another embodiment of the present invention there is provided a method for treating an HIV-1 infection, or preventing an HIV-1 infection, or treating AIDS or ARC, comprising co-administering to a host in need thereof a therapeutically effective amount of a compound according to formula I wherein R¹, R², R³, R⁴, R^{a}, R^{b}, Ar, X, X¹, Y and n are as defined herein above and a therapeutically effective amount of at least one compound selected from the group consisting of zidovudine, lamivudine, didanosine, zalcitabine, stavudine, rescriptor, sustiva, viramune, efavirenz, nevirapine, delavirdine, saquinavir, ritonavir, nelfinavir, indinavir, amprenavir, lopinavir or enfuvirtide.

In a twenty-seventh embodiment of the present invention there is provided a method for inhibiting HIV reverse transcriptase in a host infected with HIV-1 comprising administering a therapeutically effective amount of a compound according to formula I wherein R¹, R², R³, R⁴, R^{a}, R^{b}, Ar, X, X¹, Y and n are as defined herein above; or a pharmaceutically acceptable salt thereof.

In a twenty-eighth embodiment of the present invention there is provided a method for inhibiting HIV reverse transcriptase in a host infected with a strain of HIV-1 expressing a reverse transcriptase with at least one mutation compared to wild type HIV 1 said method comprising administering a therapeutically effective amount of a compound according to formula I wherein R¹, R², R³, R⁴, R^{a}, R^{b}, Ar, X, X¹, Y and n are as defined herein above; or a pharmaceutically acceptable salt thereof.

In a twenty-ninth embodiment of the present invention there is provided a method for inhibiting HIV reverse transcriptase in a host infected with a strain of HIV-1 expressing a reverse transcriptase with reduced susceptibility to efavirenz, nevirapine or delavirdine compared to wild type reverse transcriptase said method comprising administering a therapeutically effective amount of a compound according to formula I wherein R¹, R², R³, R⁴, R^{a}, R^{b}, Ar, X, X¹, Y and n are as defined herein above; or a pharmaceutically acceptable salt thereof.

In a thirtieth embodiment of the present invention there is provided a pharmaceutical composition comprising a compound according to formula I wherein R¹, R², R³, R⁴, R^{a}, R^{b}, Ar, X, X¹, Y and n are as defined herein above; or a pharmaceutically acceptable salt thereof and at least one pharmaceutically acceptable carrier, diluent or excipient. The term “wild type” as used herein refers to the HIV virus strain which possesses the dominant genotype which naturally occurs in the normal population which has not been exposed to reverse transcriptase inhibitors. The term “wild type reverse transcriptase” used herein has refers to the reverse transcriptase expressed by the wild type strain which has been sequenced and deposited in the SwissProt database with an accession number P03366.

The term “reduced susceptibility” as used herein refers to about a 10 fold, or greater, change in sensitivity of a particular viral isolate compared to the sensitivity exhibited by the wild type virus in the same experimental system.

The term “nucleoside and nucleotide reverse transcriptase inhibitors” (“NRTI”s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA. Recent progress in development of RTI and PI inhibitors has been reviewed: F. M. Uckun and O. J. D'Cruz, Exp. Opin. Ther. Pat. 2006 16:265-293; L. Menendez-Arias, Eur. Pharmacother. 2006 94-96 and S. Rusconi and O. Vigano, Future Drugs 2006 3(1):79-88.

A-M. Vandamme et al. (Antiviral Chemistry & Chemotherapy, 1998 9:187-203) disclose current HAART clinical treatments of HIV-1 infections in man including at least triple drug combinations. Highly active anti-retroviral therapy (HAART) has traditionally consisted of combination therapy with nucleoside reverse transcriptase inhibitors (NRTI), non-nucleoside reverse transcriptase inhibitors (NNRTI) and protease inhibitors (PI). These compounds inhibit biochemical processes required for viral replication. While HAART has dramatically altered the prognosis for HIV infected persons, there remain many drawbacks to the current therapy including highly complex dosing regimes and side effects which can be very severe (A. Carr and D. A. Cooper, Lancet 2000 356(9239):1423-1430). Moreover, these multidrug therapies do not eliminate HIV-1 and long-term treatment usually results in multidrug resistance, thus limiting their utility in long term therapy. Development of new therapeutics which can be used in combination with NRTIs, NNRTIs, PIs and viral fusion inhibitors to provide better HIV-1 treatment remains a priority.

Typical suitable NRTIs include zidovudine (AZT; RETROVIR®); didanosine (ddI; VIDEX®); zalcitabine (ddC; HIVID®); stavudine (d4T; ZERIT®); lamivudine (3TC; EPIVIR®); abacavir (ZIAGEN®); adefovir dipivoxil [bis(POM)-PMEA; PREVON®]; lobucavir (BMS-180194), a nucleoside reverse transcriptase inhibitor disclosed in EP-0358154 and EP-0736533; BCH-10652, a reverse transcriptase inhibitor (in the form of a racemic mixture of BCH-10618 and BCH-10619) under development by Biochem Pharma; emitricitabine [(−)—FTC] in development by Triangle Pharmaceuticals; β-L-FD4 (also called β-L-D4C and named β-L-2′,3′-dicleoxy-5-fluoro-cytidene) licensed Vion Pharmaceuticals; DAPD, the purine nucleoside, (−)-β-D-2,6-diamino-purine dioxolane disclosed in EP-0656778 and licensed to Triangle Pharmaceuticals; and lodenosine (FddA), 9-(2,3-dideoxy-2-fluoro-β-D-threo-pentofuranosyl)adenine, an acid stable purine-based reverse transcriptase inhibitor under development by U.S. Bioscience Inc.

Four NNRTIs have been approved in the USA: nevirapine (BI-RG-587; VIRAMUNE®) available from Boehringer Ingelheim (BI); delaviradine (BHAP, U-90152; RESCRIPTOR®) available from Pfizer; efavirenz (DMP-266, SUSTIVA®) a benzoxazin-2-one from BMS and etravirine (TMC-125, INTELENCE®) from Tibotec. Other NNRTIs currently under investigation include PNU-142721, a furopyridine-thiopyrimide under development by Pfizer; capravirine (S-1153 or AG-1 549; 5-(3,5-dichlorophenyl)-thio-4-isopropyl-1-(4-pyridyl)methyl-1H-imidazol-2-ylmethyl carbonate) by Shionogi and Pfizer; emivirine [MKC-442; (1-(ethoxy-methyl)-5-(1-methylethyl)-6-(phenylmethyl)-(2,4(1H,3H)-pyrimidinedione)] by Mitsubishi Chemical Co. and Triangle Pharmaceuticals; (+)-calanolide A (NSC-67545 1) and B, coumarin derivatives disclosed in NIH U.S. Pat. No. 5,489,697, licensed to Sarawak/Advanced Life Sciences; DAPY (TMC120; 4-{4-[4-((E)-2-cyano-vinyl)-2,6-dimethyl-phenylamino]-pyrimidin-2-ylamino}-benzonitrile) by Tibotec-Virco and Johnson & Johnson; BILR-355 BS (12-ethyl-8-[2-(1-hydroxy-quinolin-4-yloxy)-ethyl]-5-methyl-11,12-dihydro-5H-1,5,10,12-tetraaza-dibenzo[a,e]cycloocten-6-one by Boehringer-Ingleheim; PHI-236 (7-bromo-3-[2-(2,5-dimethoxy-phenyl)-ethyl]-3,4-dihydro-1H-pyrido[1,2-a][1,3,5]triazine-2-thione) and PHI-443 (TMC-278, 1-(5-bromo-pyridin-2-yl)-3-(2-thiophen-2-yl-ethyl)-thiourea) by Paradigm Pharmaceuticals.

Typical suitable PIs include saquinavir (Ro 31-8959; INVIRASE®; FORTOVASE®); ritonavir (ABT-538; NORVIR®); indinavir (MK-639; CRIXIVAN); nelfnavir (AG-1343; VIRACEPT®); amprenavir (141W94; AGENERASE®); TMC114 (darunavir, PREZISTA®); lasinavir (BMS-234475); DMP450, a cyclic urea under development by Triangle Pharmaceuticals; BMS-2322623, an azapeptide under development by Bristol-Myers Squibb as a 2nd-generation HIV-1 PI; ABT-378 under development by Abbott; and AG-1549 an imidazole carbamate under development by Agouron Pharmaceuticals, Inc. Additional PIs in preclinical development include N-cycloalkylglycines by BMS, α-hydroxyarylbutanamides by Enanta Pharmaceuticals; α-hydroxy-γ-[[(carbocyclic- or heterocyclic-substituted)amino)carbonyl]alkanamide derivatives; γ-hydroxy-2-(fluoroalkylaminocarbonyl)-1-piperazinepentanamides by Merck; dihydropyrone derivatives and α- and β-amino acid hydroxyethylamino sulfonamides by Pfizer; and N-aminoacid substituted L-lysine derivatives by Procyon.

Entry of HIV into target cells requires CD-4 cell surface receptor and the CCR5 (M-tropic strains) and CXCR4 (T-tropic strains) chemokine co-receptors. Chemokine antagonize which block viral binding to the chemokines are useful inhibitors of viral infection. Takeda's identified TAK-779 as a potential CCR5 antagonist. (M. Shiraishi et al., J. Med. Chem. 2000 43(10):2049-2063; M. Babba et al. Proc. Nat. Acad. Sci. USA 1999 96:5698-5703) and TAK-220 (C. Tremblay et al. Antimicrob. Agents Chemother. 2005 49(8):3483-3485). WO0039125 (D. R. Armour et al.) and WO0190106 (M. Perros et al.) disclose heterocyclic compounds that are potent and selective CCR5 antagonists. Maraviroc (UK427,857; MVC). has has been approved by the FDA for anti-HIV therapy. (P. Dorr et al., Antimicrob. Agents Chemother. 2005 49(11):4721-4732; A. Wood and D. Armour, Prog. Med. Chem. 2005 43:239-271; C. Watson et al., Mol. Pharm. 2005 67(4):1268-1282; M. J. Macartney et al., 43^{rd} Intersci. Conf. Antimicrob. Agents Chemother. Sep. 14-17, 2003, Abstract H-875). Schering has advanced Sch-351125 (SCH-C) into Phase I/II clinical studies and reported the advance of a more potent follow-up compound, Vicroviroc (Sch-417690, SCH-D) into Phase I studies. (S. W. McCrombie et al., WO00066559; B. M. Baroudy et al. WO00066558; A. Palani et al., J. Med. Chem. 2001 44(21):3339-3342; J. R. Tagat et al., J. Med. Chem. 2001 44(21):3343-3346; J. A. Esté, Cur. Opin. Investi. Drugs 2002 3(3):379-383; J. M. Struzki et al. Proc. Nat. Acad Sci. USA 2001 98:12718-12723). Merck has disclosed the preparation of (2S)-2-(3-chlorophenyl)-1-N-(methyl)-N-(phenylsulfonyl)amino]-4-[spiro(2,3-dihydrobenzothiophene-3,4′-piperidin-1′-yl)butane S-oxide (1) and related derivatives with good affinity for the CCR5 receptor and potent-HIV activity. (P. E. Finke et al., Bioorg. Med. Chem. Lett., 2001 11:265-270; P. E. Finke et al., Bioorg. Med. Chem. Lett., 2001 11:2469-2475; P. E. Finke et al., Bioorg. Med. Chem. Lett., 2001 11:2475-2479; J. J. Hale et al., Bioorg. Med. Chem. Lett., 2001 11:2741-22745; D. Kim et al., Bioorg. Med. Chem. Lett., 2001 11:3099-3102) C. L. Lynch et al. Org Lett. 2003 5:2473-2475; R. S. Veazey et al. J. Exp. Med. 2003198:1551-1562. GSK-873140 (ONO-4128, E-913, AK-602) was identified in a program initiated at Kumamoto University (K. Maeda et al. J. Biol. Chem. 2001 276:35194-35200; H. Nakata et al. J. Virol. 2005 79(4):2087-2096) and has been advanced to clinical trials. In WO00/166525; WO00/1 87839; WO02/076948; WO02/076948; WO02/079156, WO2002070749, WO2003080574, WO2003042178, WO2004056773, WO2004018425 Astra Zeneca disclose 4-amino piperidine compounds which are CCR5 antagonists. In U.S. Publication No. 20050176703 published Aug. 11, 2005, S. D. Gabriel and D. M. Rotstein disclosed heterocyclic CCR5 antagonist capable of preventing HIV cell entry. In U.S. Publication No. 20060014767 published Jan. 19, 2006, E. K. Lee et al. disclosed heterocyclic CCR5 antagonist capable of preventing HIV cell entry.

Attachment Inhibitors effectively block interaction between viral envelope proteins and chemokine receptors or CD40 protein. TNX-355 is a humanized IgG4 monoclonal antibody that binds to a conformational epitope on domain 2 of CD4. (L. C. Burkly et al., J. Immunol. 1992 149:1779-87) TNX-355 can inhibit viral attachment of CCR5—, CXCR4- and dual/mixed tropic HIV-1 strains. (E. Godofsky et al., In Vitro Activity of the Humanized Anti-CD4 Monoclonal Antibody, TNX-355, against CCR5, CXCR4, and Dual-Tropic Isolates and Synergy with Enfuvirtide, 45th Annual Interscience Conference on Antimicrobial Agents and Chemotherapy (ICAAC). Dec. 16-19, 2005, Washington D.C. Abstract # 3844; D. Norris et al. TNX-355 in Combination with Optimized Background Regime (OBR) Exhibits Greater Antiviral Activity than OBR Alone in HIV-Treatment Experienced Patients, 45th Annual Interscience Conference on Antimicrobial Agents and Chemotherapy (ICAAC). Dec. 16-19, 2005, Washington D.C. Abstract # 4020.)

Recently an integrase inhibitor, Raltegravir (MK-05 18, Merck) has been approved by the FDA and a second compound (Elvitegravir, Gilead Sciences and Japan Tabacco) is in phase II trials. Other antiviral agents include hydroxyurea, ribavirin, IL-2, IL-12, pentafuside. Hydroxyurea (Droxia), a ribonucleoside triphosphate reductase inhibitor shown to have a synergistic effect on the activity of didanosine and has been studied with stavudine. IL-2 (aldesleukin; PROLEUKIN®) is disclosed in Ajinomoto EP-0142268, Takeda EP-0176299, and Chiron U.S. Pat. Nos. RE 33,653, 4,530,787, 4,569,790, 4,604,377, 4,748,234, 4,752,585, and 4,949,314. Pentafuside (FUZEON®) a 36-amino acid synthetic peptide that inhibits fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred. Ribavirin, 1-β-D-ribofuranosyl-1H-1,2,4-triazole-3-carboxamide. CCR5 antagonists which block viral entry are also approaching approval including Maraviroc (Pfizer) and Vicriviroc (Schering).

Commonly used abbreviations include: acetyl (Ac), atmospheres (Atm), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z),1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminurnhydride.(DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), methanol (MeOH), melting point (mp), MeSO₂— (mesyl or Ms), methyl (Me), acetonitrile (MeCN), meta-chloroperbenzoic acid (MCPBA), mass spectrum (ms), methyl t-butyl ether (MTBE), N-methylmorpholine (NMM), N-methylpyrrolidone (NMP), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), triflate or CF₃SO₂— (Tf), trifluoroacetic acid (TFA), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (1-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I

| Cpd. | | | | |
|---|---|---|---|---|
| No. | structure | mw | ms | mp |
| I-1 | 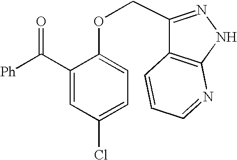 | 363.8 | | |
| I-2 | 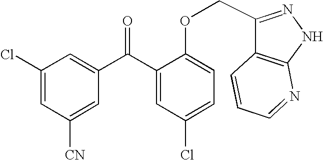 | 423.26 | 422 | 194.3-195.9 |
| I-3 | 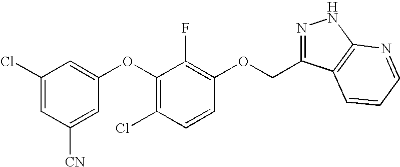 | 429.24 | 429 | |
| 1-4 | 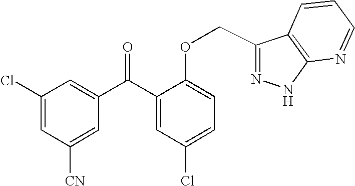 | 411.25 | | 169.6-170.0 |
| I-5 | 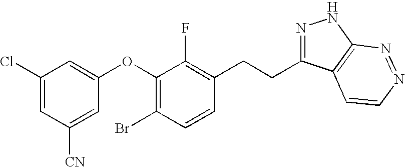 | | | |
| I-6 | 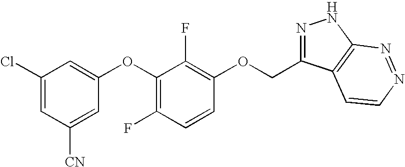 | 413.77 | 414,416 | |
| 1-7 | 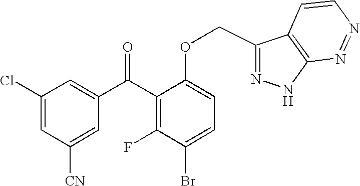 | 474.68 | 474 | |
| 1-8 | 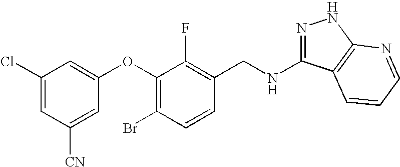 | 471.7 | 472 | |
| 1-9 | 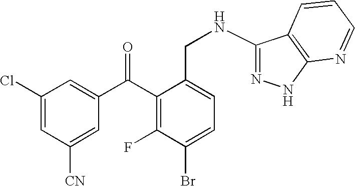 | 472 | 472&474 | 214-215 |

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2^{nd} edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

Some compounds in following schemes are depicted with generalized substituents; however, one skilled in the art will immediately appreciate that the nature and number of the R groups can varied to afford the various compounds contemplated in this invention. The general formulae in the schemes are intended to be illustrative and are not intended to imply a limitation to the scope of the invention which is defined by the appended claims. Moreover, the reaction conditions are exemplary and alternative conditions are well known. The reaction sequences in the following examples are not meant to limit the scope of the invention as set forth in the claims.

Compounds of the present invention in which the pendant pyrazole chain is meta to the aryloxy moiety are prepared (SCHEME A) from a 4-nitro-3-aryloxyphenol (A-5) which can prepared from 2,3,4-trifluoronitrobenzene or 2,4-dinitrobenzene by a two step process comprising nucleophilic aromatic displacement of the 2-fluoro by an appropriately substituted phenol and subsequent displacement of the 4-fluoro with benzaldehyde oxime under conditions which result in cleavage of the N—O bond (R. D. Knudsen and H. R. Snyder, J. Org. Chem. 1974 39(23):3343-3346). One skilled in the art will appreciate that the reaction can carried out with a variety of phenols allowing diverse substitution and regiochemistry on the aryl ring.

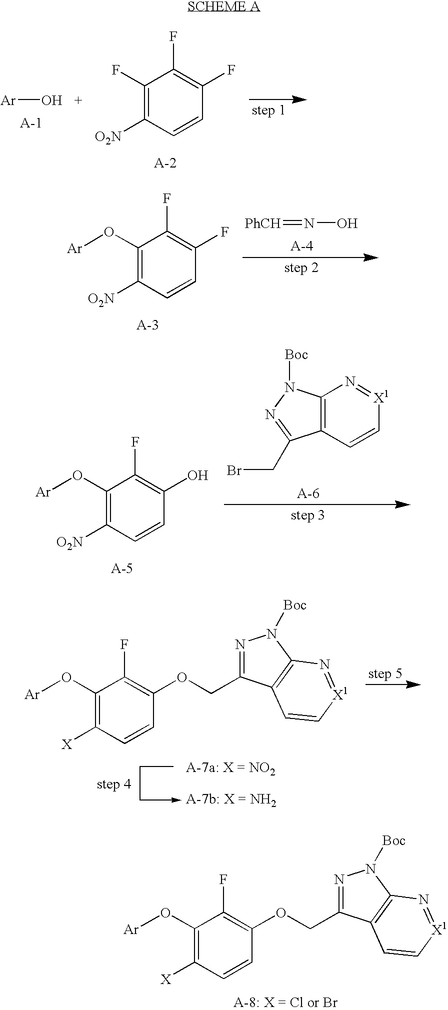

Introduction of the 1H-pyrazolo[3,4-b]pyridin-3-ylmethyl moiety (X¹═CH) was accomplished by O-alkylation of A-5 with tert-butyl 3-(bromomethyl)-1H-pyrazolo[3,4-b]pyridine-1-carboxylate (A-6, CASRN 174180-76-8). The corresponding 1H-methyl-indazole analogs can be prepared analogously from tert-butyl 3-(bromomethyl)-1H-indazole-1-carboxylate (CASRN 17418042-8).

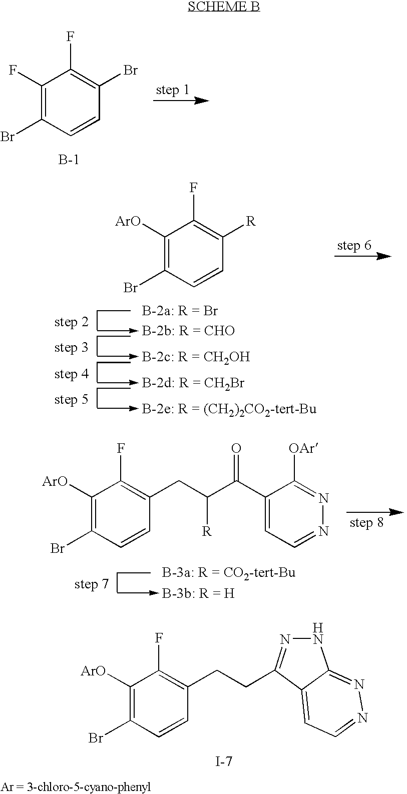

Compounds with an ethylene linker could be prepared from an appropriately substituted 2-aryl-propionic acid derivative (SCHEME B). Metallation and formylation of B-2a afforded B-2b which was reduced to the alcohol B-2c and subsequently converted to the benzyl bromide B-2d by conventional procedures. Alkylation of the anion derived from tert-butyl acetate by B-2d affords the 2-aryl-propionic ester B-2e. The requisite pyrazole precursor B-3b are assembled by Claisen condensation of B-2e and a pyridazine-4-carboxylic acid substituted with a leaving group at the 3-position. Leaving groups which have been utilized in this and related transformations include halides, sulfonate esters and substituted aryloxy ethers. A convenient protocol entails in situ activation of a heteroaryl carboxylic acid with CDI and condensation of the activated derivative with B-2e in the presence of base to afford the β-ketoester B-3a which is decarboxylated to afford B-3b.

The fused pyrazoles disclosed herein can be conveniently prepared from B-3b by an intramolecular cyclization with hydrazine or a hydrazine surrogate which can form an imine at the carbonyl center and displace the leaving group on the heteroaryl ring to form the compounds of the present invention.

Other compounds with the scope of the present invention are substituted with other halogens, alkyl or cycloalkyl moieties at the 4-position in place of the bromine. Alkyl groups can be introduced utilizing the Negishi coupling dialkylzincs with the haloarenes (E.-I. Negishi, Acc. Chem. Res. 1982 15:340-348). The reaction is catalyzed by palladium Pd(0) and palladium is preferably ligated to a bidentate ligand including Pd(dppf)Cl₂ and Pd(dppe)Cl₂. (J. M. Herbert Tetrahedron Lett. 2004 45:817-819) Typically the reaction is run an inert aprotic solvent and common ethereal solvents include dioxane, DME and THF are suitable. The reaction is commonly run at elevated temperature. The Negishi reaction was utilized to introduce methyl and ethyl substituents.

The 4-cyclopropyl substituent is introduced in two steps employing a Stille palladium-mediated coupling of ethenyltrimethyltin and an aryl bromide and subsequently cyclopropanation of the resulting olefin. The cyclopropanation was accomplished Pd(OAc)₂ catalyzed cycloaddition of diazomethane. Other cyclopropanation conditions are well known in the art and could be adapted to this substrate.

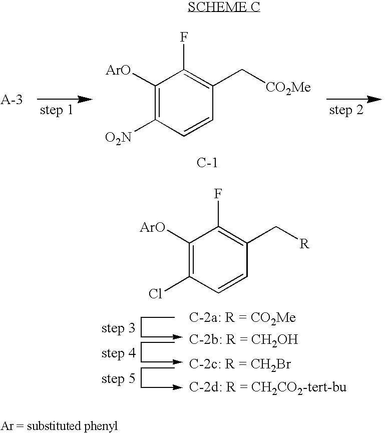

Compounds of the present invention with a two-atom ethylene linker and a chlorine substituent in place of the bromine substitutent can be conveniently prepared from A-3 by displacing the fluorine substituent para to the nitro group with tert-butyl methyl malonate which is decarboxylated to afford the corresponding phenylacetic acid ester C-1 (SCHEME C). Details of this methodology have been disclosed by D. J. Kertesz et al. in U.S. Patent Pub. 2005/0234236 published Oct. 20, 2005 which is hereby incorporated by reference in its entirety. Conversion of C-2d to the fused pyrazoles of the present invention is carried out as depicted in SCHEME B.

The nitro substituent in C-1 affords an alternative route to introduce other ring substituents by reduction to the corresponding amine which can be diazotized and displaced by a variety of nucleophiles. Reduction of the nitro group can be carried out with a variety of well-known reducing agents. For example an activated metal such as activated iron, zinc or tin (produced for example by washing iron powder with a dilute acid solution such as dilute hydrochloric acid). The reduction can also be carried out under a hydrogen atmosphere in the presence of an inert solvent in the presence of a metal effective to catalyze hydrogenation reactions such as platinum or palladium. Other reagents which have been used to reduce nitro compounds to amines include AlH₃—AlCl₃, hydrazine and a catalyst, TiCl₃, Al—NiCl₂-THF, formic acid and Pd/C and sulfides such as NaHS, (NH₄)₂S or polysulfides (i.e. the Zinn reaction). Aromatic nitro groups have been reduces with NaBH₄ or BH₃ in the presence of catalysts such as NiCl₂ and CoCl₂. Thus for example, reduction may be effected by heating the nitro group in the presence of a sufficiently activated metal such as Fe and a solvent or diluent such as H₂O and alcohol, for example MeOH or EtOH at a temperature in the range of 50 to 150° C., conveniently at about 70° C. (J. March, Advanced Organic Chemistry, John Wiley & Sons: New York, N.Y., 1992, p 1216).

Conversion of the aryl amine to an aryl halides was carried out by diazotization of the amine and displacement of the resulting diazonium group with a halide were carried out under standard Sandmeyer conditions. Diazotization of the aryl amines is accomplished by treating the amine with nitrous acid which is commonly formed by treating an solution of the amine in dilute HCl with an aqueous solution of sodium nitrite at 0-10° C. Other mineral acids such as sulfuric acid and phosphoric acid can be used if the chloride counterion is undesirable. Diazotization of amines can be carried out in organic solvents such as HOAc, MeOH, EtOH, formamide and DMF in the presence of nitrous acid esters such as. butyl nitrite and pentyl nitrite. (K. Schank, Preparation of diazonium groups, In The chemistry of diazonium and diazo groups, Part 2; S. Patai, Ed.; John Wiley & Sons: New York, N.Y., 1978, p. 647-648). Conversion of the resulting diazonium salt to a chlorine or bromine is carried out in HCl/Cu(I)Cl or HBr/Cu(I)Br. Aryl bromide and chlorides can also be prepared from primary aromatic amines by treating the amine with tert-butyl nitrite and anhydrous CuCl₂ or CuBr₂ at 65° C. or with tert-butyl thionitrite or tert-butyl-thionitrate and CuCl₂ or CuBr₂ at RT. (J. March, Advanced Organic Chemistry, John Wiley & Sons: New York, N.Y., 1992, p 723)

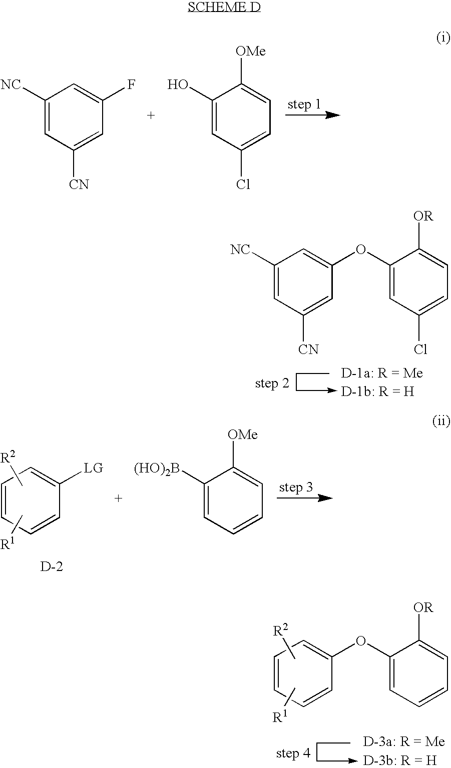

2-Aryloxy-phenols are precursors to compounds of the present invention in which the pendant fused pyrazole moiety is ortho to aryloxy moiety. 2-Aryloxy-phenols can be prepared by methodology known in the art (SCHEME D). The preparation of diaryl ethers has been reviewed (J. S. Sawyer, Recent Advances in Diaryl Ether Synthesis, Tetrahedron 2000 56:5045-5065). Introduction of the (hetero)aryloxy ether can often be accomplished by direct S_{N}Ar displacement reaction on a aromatic ring substituted with a leaving group and electronegative substituents. In the present example direct displacement of a compound with a leaving group, e.g. 3-fluoro-iso-phthalonitrile [CASRN 453565-55-4] by guiacol and subsequent demethylation of the resulting phenol will afford the useful intermediates such as D-1b. Other aryl fluorides also useful for compounds of the present invention include, but are not limited to, 3-chloro-5-fluoro-benzonitrile [CASRN 327056-73-5], 3-difluoromethyl-3-fluoro-benzonitrile [CASRN 867366-77-6] and 3,5-difluoro-benzonitrile [CASRN 64248-63-1]. (L. H. Jones and C. Mowbray, Syn. Lett. 2006, No. 9:1404-1406)

Aryl ethers also can be efficiently prepared by Cu(OAc)₂ catalyzed condensation of substituted benzene boronic acids and phenols as depicted in reaction (ii) of SCHEME D (D. A. Evans et al., Tetrahedron Lett., 1998 39:2937-2940 and D. M. T. Chan et al., Tetrahedron Lett. 1998 39:2933-2936). Benzene boronic acids with a variety of other substituents are widely available. Alternatively, variations of the Ullmann diaryl ether synthesis with Cu(I) salts (J.-F. Marcoux et al., J. Am. Chem. Soc. 1997 119:10539-540; E. Buck et al, Org. Lett. 2002 4(9): 1623-1626) or palladium-catalyzed coupling procedures also has been reported (G. Mann et al., J. Am. Chem. Soc., 1999 121:3224-3225) have been described. These protocols do not require strongly electronegative substituents to activate an aryl fluoride for S_{N}Ar displacements. One skilled in the art will appreciate that optimal procedure will vary depending on the nature and position of substituents on the aryl rings to be coupled and useful conditions for the coupling can by identified without undue experimentation.

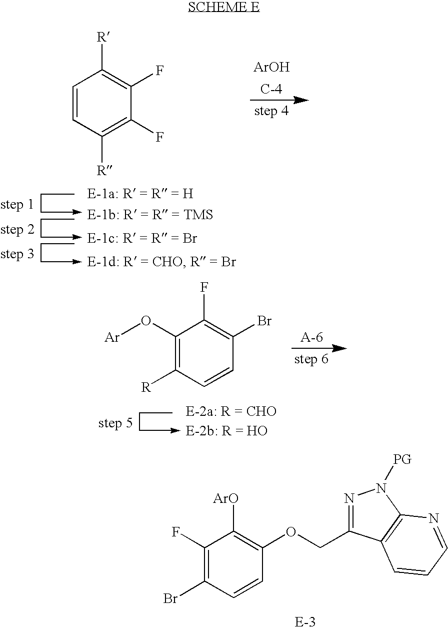

An alternate route (SCHEME E) leading to compounds of the present invention in which the pendant pyrazole chain is ortho to aryloxy moiety utilizes the ortho fluoro benzaldehyde derivative E-1d which was treated with a suitably substituted phenol resulting in displacement of the fluorine ortho to formyl substituent. Baeyer-Villager oxidation and subsequent hydrolysis of the formate ester converts the formyl group to a phenol which can be converted into a pyrazole by alkylation with A-6 as depicted in SCHEME A.

Analogs with a pendant 1H-pyrazolo[3,4-c]pyridazin-3-yl-methyl were prepared by an insertion reaction of carbene derived from 2-diazo-1-[3-(2,4-difluoro-phenoxy)-pyridazin-4-yl]-ethanone into the O—H bond of the phenol to afford a ketone (e.g. 50, example 9, infra) which can be cyclized with hydrazine or by converting the diazoketone to the alpha-chloro-ketone (e.g., 52, example 10) which can be used to alkylate a phenol and afford the requisite ketone.

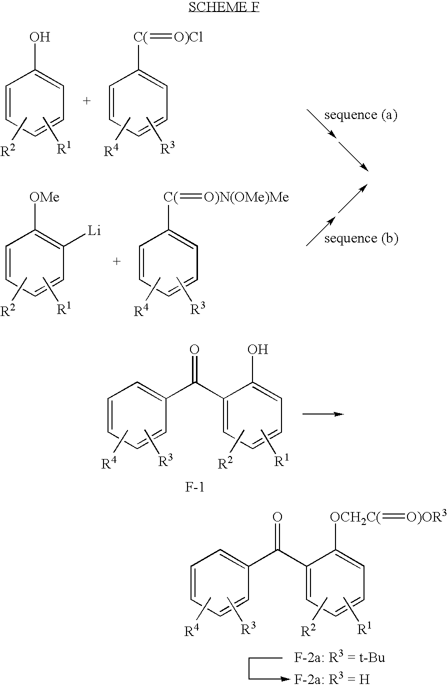

The preparation of 2-aroyl-phenol derivatives F-1 which are precursors to compounds of the present invention in which R² is ArC(═O) an be prepared by acylation of a substituted phenol with an substituted aroyl chloride followed by a Fries rearrangement (sequence a) or by ortho-metallation of an anisole derivative and condensation with a suitably substituted N,O-dimethyl-N-hydroxy-benzamide (sequence b) as depicted in SCHEME F. (P. G. Wyatt et al., J. Med. Chem. 1995 38(10):1657-1665; J. H. Chan et al., J. Med Chem. 2004 47(5):1175-1182; K. Romines et al., J. Med. Chem. 2006 49(2):727-739; C. W. Andrews et al. WO01/017982 published Mar. 15, 2002; and J. H. Chan et al. WO02/070470, published Sep. 12, 2002) These references are hereby incorporated by reference in their entirety. Conversion of F-1 to compounds claimed herein can be accomplished by directly alkylating the phenol with a bromomethyl-pyrazole derivative A-6 as depicted in SCHEME A or by alkylating the phenol with an acetic acid derivative which can undergo the Claisen/intramolecular cyclization sequence depicted in SCHEME B.

### Dosage and Administration

The compounds of the present invention may be formulated in a wide variety of oral administration dosage forms and carriers. Oral administration can be in the form of tablets, coated tablets, dragees, hard and soft gelatine capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” the substance is useful in preparing a pharmaceutical composition that is generally safe, non-toxic, and neither biologically nor otherwise undesirable and includes that which is acceptable for human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfinuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

In embodiments of the invention, the active compound or a salt can be administered in combination with another antiviral agent, such as a nucleoside reverse transcriptase inhibitor, another nonnucleoside reverse transcriptase inhibitor or HIV protease inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent. In embodiments of the invention, the active compound or a salt can be administered in combination with another antiviral agent, such as a nucleoside reverse transcriptase inhibitor, another nonnucleoside reverse transcriptase inhibitor or HIV protease inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

It will be understood that references herein to treatment extend to prophylaxis as well as to the treatment of existing conditions, and that the treatment of animals includes the treatment of humans as well as other animals. Furthermore, treatment of a HIV-1 infection, as used herein, also includes treatment or prophylaxis of a disease or a condition associated with or mediated by HIV-1 infection, or the clinical symptoms thereof.

### REFERENTIAL EXAMPLE 1

### Phenols

### Preparation of 3-chloro-5-hydroxy-benzonitrile (CASRN 473923-97-6)

step 1—A 100 ml round bottom flask was charged under a stream of nitrogen with 3,5-dichlorobenzonitrile (R-3a, 7.0 g, 40.69 mmol) and anhydrous DMF (75 mL). To the solution was added sodium methoxide (2.26 g, 44.76 mmol) and resulting solution was stirred further at RT for 24 h. When the reaction was complete, aqueous 10% HCl added dropwise to the reaction vessel. The crude mixture was extracted with EtOAc and sequentially washed with aqueous acid, water and brine. The EtOAc extracts were dried (Na₂SO₄), filtered and the solvent was removed in vacuo to afford a crude solid which was recrystallized from hexane/acetone to afford 5.9 g (86%) of 5-chloro-3-methoxy-benzonitrile.

step 2—A 250 mL flask was charged with 5-chloro-3-methoxy-benzonitrile (7.0 g, 41.766 mmol) and 2,4,6-collidine (100 μL). The mixture was heated to 170° C. and LiI (16.76 g, 125.298 mmol) was added and the reaction mixture was heated for 4 h. When R-3b was consumed the reaction was cooled to RT and quenched with 10% aqueous HCl. The resulting mixture was extracted with EtOAc and washed with water and brine. The EtOAc extract was dried over (Na₂SO₄) and filtered. The solvent was removed in vacuo to afford a yellow oil which was purified by silica gel chromatography eluting with EtOAc/hexane (10:90) to afford 6.0 g (94%) of 3-chloro-5-hydroxy-benzonitrile.

### Preparation of 5-hydroxy-isophthalonitrile [CASRN 79370-78-8]

5-Hydroxy-isophthalonitrile was prepared as described by C. E. Mowbary et al., WO2004024147 published Mar. 25, 2004 in procedures 1-3.

### Preparation of 3-cyano-5-difluoromethyl-phenol [CARN 874974-85-3]

step 1—A solution of 1,3-dibromo-5-fluoro-benzene (CASRN 1435-514), MeONa (1 equivalent) and DMF were stirred overnight under an N₂ atmosphere at RT. The volatile solvents were removed in vacuo and the residue partitioned between Et₂O and water. The organic phase was washed with 5% NaOH, water and brine, dried (MgSO₄), filtered and evaporated to afford 1,3-dibromo-5-methoxy-benzene.

step 2—To a solution of 1,3-dibromo-5-methoxy-benzene (60 g, 0.2256 mol) and anhydrous Et₂O (1 L) cooled to −78° C. and maintained under an Ar atmosphere was added dropwise over 30 min n-BuLi (100 mL, 0.2482 mol, 2.5M in hexane). The yellow solution was stirred at −78° C. for 20 min. To the reaction mixture was added dropwise dry DMF (19 mL, 248.2 mmol) over 15 min and the reaction stirred at −78° C. for 10 min before the cooling bath was removed and the reaction allowed to warm to −30° C. over 30 min. The reaction vessel was placed in an ice-water bath and warmed to −10° C. The mixture was slowly added to an ice cold saturated aqueous NH₄Cl solution (400 mL). The organic layer was separated and the aqueous phase thrice extracted with Et₂O. The combined extracts were washed with water, dried (MgSO₄), filtered and evaporated to afford an oil which solidified on standing. The crude product was purified by SiO₂ chromatography eluting with a hexane/EtOAc gradient (3 to 5% EtOAc) to afford 3-bromo-5-methoxy-benzaldehyde.

step 3—A solution of 3-bromo-5-methoxy-benzaldehyde (1 mmol) in DMF (2 mL) is added to a round bottomed flask containing Zn(CN)₂ (0.7 equivalents), Pd(PPh₃)₄(0) (0.2 equivalents) in DMF (15 mL). The reaction is stirred at 90° C. under an atmosphere of argon for 48 h. The reaction mixture is cooled and evaporated to dryness. The crude residue is dissolved in EtOAc, washed with brine solution, dried (MgSO₄) and evaporated. The crude product is purified by SiO₂ chromatography to afford 3-formyl-5-methoxy-benzonitrile.

step 4—DAST (21.04 mL, 519 mmol) was added to a solution of 3-formyl-5-methoxy-benzonitrile (15.1 g, 94 mmol) and DCM (100 mL) contained in a NALGENE® bottle under nitrogen. EtOH (0.013 mL, 0.23 mmol) was added, and the mixture was stirred for 16 h. The reaction mixture was then added slowly to an aqueous solution of saturated NaHCO₃. After the bubbling was ceased, DCM (50 mL) was added and the layers were separated. The organic layer was washed with brine (30 mL) and dried (MgSO₄). The solvent was removed and the crude product was purified by two flash SiO₂ chromatographies eluting with an EtOAc/hexanes gradient (0% to 10% EtOAc) to 3-difluoromethyl-5-methoxy-benzonitrile as a white solid.

step 5-3-Difluoromethyl-5-methoxy-benzonitrile was demethylated in a solution of 48% aqueous HBr and glacial HOAc heated to 120° C. until demethylation was complete. Removal of volatile solvents and partitioning between water and DCM afforded 3-difluoromethyl-5-hydroxy-benzonitrile.

### Preparation of 3-bromo-5-cyano-phenol (CASRN 770718-92-8)

step 1—n-BuLi (2.6 mL of a 1.6 M solution, 1.1 equiv) was added slowly to a solution of the 1,3-dibromo-5-methoxy-benzene (1.0 g, 3.8 mmol, CAS Reg. No. 74137-36-3) in Et₂O (20 mL) cooled to −78° C. under an N₂ atmosphere. The solution was stirred for 45 min, and DMF was added via syringe. The solution was warmed slowly to RT, added to saturated ammonium chloride, and extracted with ether. The organic phase was washed with brine and dried (MgSO₄), filtered and evaporated to afford 0.80 g (98%) of 1-bromo-3-formyl-benzaldehyde.

step 2—A solution of 1-bromo-3-formyl-benzaldehyde (12.0 g, 56 mmol), hydroxylamine hydrochloride (19.4 g, 5 equiv), EtOH (100 mL) and pyridine (10 mL) was heated to 65° C. for 16 h. The mixture was cooled to RT, and partitioned between 50% EtOAc/hexanes and water. The organic layer was washed with brine and dried (MgSO₄). The volatile materials were evaporated to afford 12.4 g (97%) of the oxime. This material was dissolved in anhydrous dioxane (100 mL) and pyridine (26 mL, 6 equiv). The solution was cooled to 0° C., TFAA (15 mL, 2 equiv) was added, and the mixture was allowed to warm to RT. The solution was stirred for 2 d, and warmed to 60° C. for 1 h. The mixture was cooled to RT, and added carefully to ice water. The mixture was extracted with DCM, and the combined organic layers were washed with water, 1 M HCl, and brine. The organic layer was dried (MgSO₄) and evaporated to afford 10.4 g (90%) of 3-bromo-5-methoxy-benzonitrile.

step 3—Anhydrous collidine (100 mL) was added to a dry flask containing 3-bromo-5-methoxy-benzonitrile (10.4 g, 49 mmol) and LiI (19.6 g, 3 equiv). The solution was heated under nitrogen to 150° C. overnight, cooled to RT, and poured into an ice cold 1 M HCl solution. The mixture was extracted with a 1:1 EtOAc/hexanes solution, washed with water, and dried (MgSO₄). Concentration in vacuo afforded 8.7 g (89%) of 3-bromo-5-hydroxy-benzonitrile.

### EXAMPLE 1

### 3-Chloro-5-[6-chloro-2-fluoro-3-(1H-pyrazolo[3,4-b]pyridin-3-ylmethoxy)-phenoxy]-benzonitrile, trifluoroacetate salt (1-3) (Scheme A)

step 1—Solid KOtBu (9.7 g, 1.05 equiv) was added to a solution of A-1 (Ar=3-chloro-5-cyano-phenyl, 12.7 g, 83 mmol) in THF (350 mL) at 0° C. The mixture was stirred for 20 min and A-2 (10 mL, 1.05 equiv) was added. The solution was warmed to RT and aged for 2 h. The mixture was poured into an aqueous ammonium chloride solution and extracted with EtOAc. The organic layer was dried (MgSO₄), filtered and the volatile materials were evaporated. Recrystallization of the resulting solid from MeOH afforded A-3.

step 2—To dry DMSO (125 mL) was added NaH (3.6 g of a 55% suspension, 2.1 equiv) and the resulting suspension was heated to 70° C. for 30 min. The solution was briefly removed from heating bath, and the benzaldoxime (9.5 g, 2 equiv) was added dropwise. The mixture was stirred at 70° C. for an additional 30 min. The thick yellow solution was cooled to RT, and a solution of A-3 (Ar=3-chloro-5-cyano-phenyl, 12.2 g, 39 mmol) and DMSO (100 mL) was added dropwise. The mixture was heated until the reaction solution became homogenous. The reaction mixture was stirred at RT for 2 h then poured into water. The resulting mixture was extracted with Et₂O, dried and evaporated to afford A-5 as a solid that was recrystallized from MeOH (8.5 g, 70%).

steps 3 & 4—To a solution of A-6 (X¹═CH, 0.25 g, 1 equiv) and A-5 (Ar=3-chloro-5-cyano-phenyl, 0.25 g, 0.8 mmol) in acetone (4 mL) was added K₂CO₃ (0.22 g, 2 equiv) and the solution was heated to 50° C. for 4 h. The reaction mixture was cooled, poured into water, and the aqueous layer was extracted with EtOAc, dried (MgSO₄), filtered and concentrated to afford 0.44 g of A-7a as a brown oil which was used without further purification. A THF solution of sulfated Pd/C (100 mg), vanadyl acetylacetonate (34 mg) and the A-7a was stirred under a H₂ atmosphere for 30 h. The mixture was filtered through CELITE® and washed with DCM and the solvent evaporated. The crude product was purified by SiO₂ chromatography eluting with an EtOAc/hexane gradient (50% to 100% EtOAc) to afford 0.11 g (25%) of A-7b (Ar=3-chloro-5-cyano-phenyl).

step 5—To a solution of the A-7b (0.11 g, 0.2 mmol) in MeCN (1 mL) at 60° C. was added a mixture of t-BuONO (0.03 mL, 1.3 equiv) and CuCl₂ (0.04 g, 1.3 equiv) in MeCN (3 mL). After 3 h, the reaction mixture was cooled to RT, quenched with aqueous NH₄Cl, and the aqueous layer was extracted with EtOAc. The combined organic extracts were dried (MgSO₄), filtered, and concentrated. The crude product was purified by reverse phase HPLC to afford 0.02 g (20%) of I-3.

### EXAMPLE 2

### 3-Chloro-5-[5-chloro-2-(1H-pyrazolo[3,4-b]pyridin-3-ylmethoxy)-phenoxy]-benzonitrile (I-4)

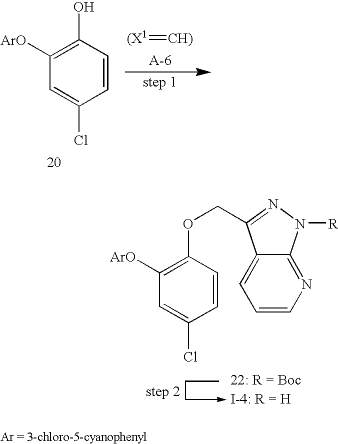

step 1—To a solution of 20 (CASRN 895572-24-4, 0.15 g, 0.54 mmol) and A-6 (X¹═CH, 0.17 g, 1 equiv) in acetone (2 mL) was added K₂CO₃ (0.18 g, 2.5 equiv) and the resulting solution was heated to 50° C. for 2 h, cooled, and evaporated. The residue was partitioned between EtOAc and aqueous NH₄Cl. The organic layer was washed with brine, dried, filtered and evaporated to afford 22 which was used without additional purification.

step 2—To a solution of 22 and dioxane (1 mL) was added a solution of 4 M HCl (1 mL). The solution was stirred overnight, diluted with DCM, and poured into saturated aqueous NaHCO₃. The aqueous layer was extracted with DCM, and the organic phases were dried (MgSO₄), filtered and evaporated. The crude product was purified by SiO₂ chromatography eluting with an EtOAc/hexane gradient (10 to 50% EtOAc) to afford 0.100 g (44%) of I-4.

3-Chloro-5-[5-chloro-2-(1H-pyrazolo[3,4-c]pyridazin-3-ylmethoxy)-phenoxy]-benzonitrile (1-7) was prepared analogously except in step 1,3-bromomethyl-pyrazolo[3,4-b]pyridine-1-carboxylic acid tert-butyl ester was replaced with 3-bromomethyl-pyrazolo[3,4-c]pyridazine-1-carboxylic acid tert-butyl ester

### EXAMPLE 3

### 3-Chloro-5-[5-chloro-2-(1H-pyrazolo[3,4-b]pyridin-3-ylmethoxy)-benzoyl]-benzonitrile (I-2)

step 1—A flask was charged with 3-chloro-5-(5-chloro-2-hydroxybenzoyl)-benzonitrile (CASRN 329944-65-2, 0.075 g, 0.258 mmol), A-6 (X¹═CH, 0.08 g, 1 eq.) and K₂CO₃ (0.07 g, 2 eq.) and flushed with nitrogen. Acetone (1 mL) was added and the reaction was heated to 60° C. for 2 h. The reaction mixture was cooled and then extracted with EtOAc, washed with water and brine. The organic layer was dried (Na₂SO₄), filtered, and evaporated. The crude product was purified by SiO₂ chromatography eluting with an EtOAc/hexane gradient (5% to 30% EtOAc) to afford 0.100 g (72%) of tert-butyl 3-[4-chloro-2-(3-chloro-5-cyano-benzoyl)-phenoxymethyl]-pyrazolo[3,4-b]pyridine-1-carboxylate (24) as a white solid.

step 2—To a solution of 24 (0.5 g, 0.955 mmol) dissolved in dioxane (4.2 mL) was added dropwise HCl (2.39 mL of 4M in dioxane, 10 eq.). The reaction was stirred at RT for 18 h then saturated aqueous NaHCO₃ was added. The aqueous solution was extracted with MeOH/DCM and the combined extracts were evaporated. The crude product was purified by SiO₂ chromatography eluting with an EtOAc/hexane gradient (15%-50% EtOAc) to afford 0.330 g (82%) of 1-2 as a white powder.

### EXAMPLE 4

### [5-Chloro-2-(1H-pyrazolo[3,4-b]pyridin-3-ylmethoxy)-phenyl]-phenyl-methanone (I-1)

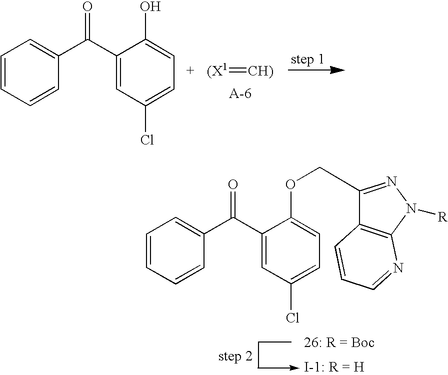

step 1—A flask was charged with (5-chloro-2-hydroxy-phenyl)-phenyl-methanone (CASRN 85-19-8, 0.05 g, 0.215 mmol), A-6 (X¹═CH, 0.067 g, 1 eq.), and K₂CO₃ (0.06 g, 2 eq.) and flushed with nitrogen. Acetone (1 mL) was added, and the reaction was heated to 50° C. for 4 h, then at 30° C. for 12 h. The reaction mixture was cooled, extracted with EtOAc, washed with water and brine. The organic layer was dried (Na₂SO₄), filtered and evaporated. The crude product was purified by SiO₂ chromatography eluting with EtOAc/hexane gradient (5% to 35% EtOAc) to afford 0.070 g (70%) of 26 as a white solid.

step 2—To a solution of 26 (0.07 g, 0.151 mmol) and dioxane (3 mL) was added dropwise HCl (0.4 mL of 4M in dioxane, 10 eq.) and the resulting solution was stirred at RT for 18 h. Aqueous saturated NaHCO₃ was added to the reaction mixture. The aqueous solution was extracted with MeOH/DCM and the organic layer was evaporated. The crude product was purified by SiO₂ chromatography eluting with a MeOH/DCM gradient (0% to 10% MeOH) to afford 0.025 g (45%) of I-1.

### EXAMPLE 5

### 3-{6-Bromo-2-fluoro-3-[2-(1H-pyrazolo[3,4-c]pyridazin-3-yl)-ethyl]-phenoxy}-5-chloro-benzonitrile (I-5)

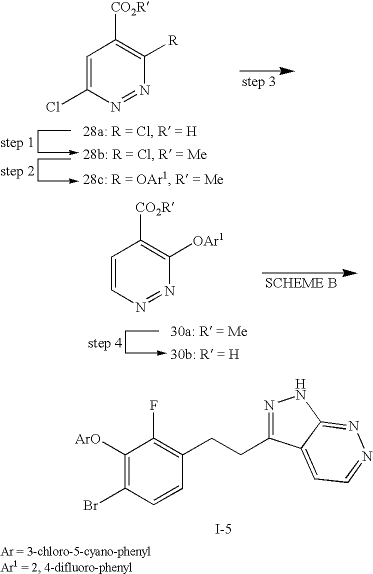

### Preparation of 3-(2,4-difluoro-phenoxy)-pyridazine-4-carboxylic acid (30b)

step 1—To a solution of 28a (7.5 g, 38.9 mmol, Aldrich) in DCM (30 mL) and MeOH (10 mL) cooled to 0° C. was added slowly via pipette, a solution of (trimethylsilyl)diazomethane (2.0 M in hexane) until a persistent yellow color is observed. After addition was complete, the solvents were removed in vacuo. The crude product was purified by SiO₂ chromatography eluting with an EtOAc/hexane gradient (10 to 25% EtOAc) to afford 3.89 g (86%) of 28b as a brown oil that solidifies on standing.

step 2—Sodium hydride (1.53 g, 38.27 mmol) was suspended in dry THF (70 mL) under a N₂ atmosphere, cooled to 0° C. and 2,4-difluorophenol (3.31 mL, 34.94 mmol) was added dropwise, via syringe. After the addition was complete the mixture was stirred for 15 min, then the cooling bath was removed for 30 min and finally the solution was again cooled to 0° C. A solution of 28b (6.89 g, 33.28 mmol) in dry THF (20 mL) was added through a cannula. The resulting mixture was stirred at RT overnight and then heated to 50° C. for 3 h. The reaction was cooled to RT and saturated NH₄Cl (40 mL) was added followed by water (60 mL). The mixture was thrice extracted with EtOAc, dried (MgSO₄), filtered and evaporated. The crude product was purified by SiO₂ chromatography eluting with an EtOAc/hexane gradient (10 to 20% EtOAc) to afford 8.15 g (82%) of 28c as a light yellow oil.

step 3—To a solution of 28c (8.15 g, 127.1 μmol) in MeOH (40 mL) was added ammonium formate (8.55 g, 1.1 eq) followed by 10% Pd—C (500 mg). The mixture was heated to 50° C. for 20 min and then to 60° C. for 35 min. The mixture was cooled to RT and filtered through a 2 cm plug of CELITE® which was rinsed well with MeOH. The volatile solvents were evaporated and the residual material partitioned between DCM (80 mL) and H₂O. The DCM layer was separated and the aqueous layer extracted twice with DCM and water (80 mL). The combined extracts were dried (MgSO₄), filtered and evaporated. The crude product was purified by SiO₂ chromatography eluting with an EtOAc/hexane gradient (10 to 50% EtOAc) to afford 5.5 g (76%) of 30a as a semi-viscous yellow oil.

step 4—To a solution of 30a (5 g, 18.78 mmol) in THF (40 mL) and MeOH (10 mL) was added an aqueous solution of LiOH (21.6 mL, 1 M solution). The mixture was stirred for 15 min when the reaction was complete as determined by TLC analysis. The mixture was concentrated and the residue was diluted with H₂O (25 mL) and THF (20 mL) and then adjusted to pH 2-3 with 10% HCl. The resulting solid was collected by filtration, washed with water (50 mL) and EtOAc (30 mL) to obtain 4.08 g (86%) of 30b as a white powder.

### Preparation of tert-butyl 3-[4-Bromo-3-(3-chloro-5-cyano-phenoxy)-2-fluoro-phenyl]-propionate (Step Numbers Refer to Scheme B)

step 1—To a solution 3-chloro-5-hydroxy-benzonitrile (153 mg, 1 mmol) and DMA (1 μL) was added NaH (42 mg, 1.05 equiv., 60% mineral oil dispersion) and the resulting mixture was stirred at 50° C. for 30 min. To the solution was added B-1 (2.7 g, 10 mmol) and the resulting mixture was heated at 125° C. for 2 h. The solution was cooled and diluted with EtOAc and the resulting solution washed with an equal volume of 10% H₂SO₄. The organic extract was dried (MgSO₄), filtered and concentrated in vacuo. The crude product was purified by SiO₂ chromatography eluting with 10% EtOAc/hexane to afford 331 mg (82%) of B-2a (Ar=3-chloro-5-cyano-phenyl).

step 2—To a solution of B-2a (2.00 g, 4.93 mL) in PhMe (40 mL) maintained under an Ar atmosphere and cooled to −78° C. was added a solution of i-PrMgCl (2M in THF, 3.08 mL, 6.16 mmol). The solution was stirred for 1 h then a solution of CuCN.2LiCl (1 M in THF, 0.1 mL) was added. The resulting solution was stirred at −50° C. for 2 h and then the reaction mixture was cannulated into a flask containing DMF (0.57 mL, 7.4 mmol) and PhMe (10 1 mL) which was cooled to −78° C. The mixture was warmed to RT and quenched by the addition of saturated aqueous NH₄Cl solution. The organic phase was separated, washed with brine, dried (MgSO₄) and evaporated to dryness in vacuo to afford 1.50 g (86%) of B-2b as an off-white solid.

step 3—Sodium borohydride was added in portions to a stirred solution of B-2b in THF (5 mL) and MeOH (5 mL) at RT. After stirring for 24 h, the reaction mixture was quenched by the addition of saturated aqueous NH₄Cl. The organics were extracted with EtOAc, washed with brine, dried (MgSO₄) and evaporated to dryness under in vacuo. The product was purified by SiO₂ chromatography eluting with an EtOAc/hexane gradient (10 to 50% EtOAc) to afford 0.25 g (31%) of B-2c.

step 4—To a stirred solution of B-2c (3.00 g, 8.41 mmol) in DCM (100 mL) was added a solution of PBr₃ (1M in DCM, 9.3 mL). After stirring at RT under N₂ for 24 h the reaction mixture was quenched by the addition of saturated aqueous NaHCO₃. The organic phase was separated, washed with brine, dried (MgSO₄) and evaporated in vacuo. The product was purified by SiO₂ chromatography eluting with an EtOAc/hexane gradient (20 to 50% EtOAc) to afford 2.0 g (57%) of B-2d as white crystals.

step 5—To a solution of diisopropylamine (1.18 mL, 1 equiv) in THF (20 mL) cooled to 0° C. was added n-BuLi (5.48 mL of a 1.6 M solution in hexanes, 1 equiv). The solution was cooled to −78° C., and tert-butyl acetate (1.18 mL, 1 equiv) was added. The solution was aged for 30 min, warmed to −50° C., and a solution of B-2d (3.6 g, 8.8 mmol) in THF (10 mL) was added. The reaction mixture was slowly warmed to RT and quenched with aqueous NH₄Cl. The aqueous layer was extracted with EtOAc, and the combined organic extracts were dried, filtered and concentrated to afford 3.8 g (96%) of B-2e as a yellow oil that was used without further purification.

step 6—To a solution of B4 (605 mg, 2.4 mmol) in DMF (10 mL) is added CDI (410 mg, 2.5 mmol). The mixture is heated to 50° C. under an Ar atmosphere for 1.5 h. The solution is cooled to −10° C. and a solution of B-2e (1.13 g, 2.5 mmol) in DMF (5 mL) is added via syringe. While stirring vigorously, NaH (336 mg, 8.4 mmol, 60% mineral oil dispersion) is added in 3 portions over 20 min. The orange solution is stirred for another 10 min and then the cooling bath is removed. The mixture is stirred for 1 h at RT. The reaction mixture is diluted with saturated NH₄Cl (20 mL), water (30 mL) and EtOAc (50 mL) and agitated. The EtOAc phase is washed with brine (50 mL) and the brine solution is extracted with EtOAc (2×30 mL). The combined extracts are dried (MgSO₄), filtered and evaporated. The crude product is purified by SiO₂ chromatography eluting with an EtOAc/hexane gradient to afford B-3a.

step 7—To a solution of B-3a (670 mg, 1.06 mmol) in DMSO (8 mL) is added water (0.4 mL) and brine (10 drops). The mixture is heated to 145° C. (oil bath temperature) under Ar atmosphere for 10 min. The solution is cooled to RT and water (60 mL), EtOAc (30 mL) and Et₂O (30 mL) are added. The mixture was agitated and NaCl (2 gm) is added. The mixture is again agitated and the organic phase is collected, washed with brine solution (50%) and the brine solution is back-extracted with EtOAc/Et₂O (1:1, 2×50 mL). The combined organic phases are dried (MgSO₄), filtered and evaporated. The crude product is purified by preparative TLC developing with EtOAc/hexanes to afford B-3b.

step 8—To a solution of B-3b (100 mg, 0.17 mmol) in MeOH (2 mL) is added sequentially tert-butyl carbazate (45 mg, 2 eq) and glacial HOAc (0.03 mL). The mixture is heated at 60° C. for 5 h and then is stirred at RT overnight. The mixture is partitioned between DCM (20 mL) and 5% NaHCO₃ (20 mL). The aqueous phase is back-extracted with DCM (2×20 mL) and the combined organic extracts are dried (MgSO₄), filtered and evaporated. This residue is dissolved in THF (4 mL) in a microwave vial, DBU (0.04 mL, 1.5 equivalents) is added and the resulting solution is heated for 10-12 min at 150° C. in microwave. The mixture is partitioned among EtOAc (40 mL), water (30 mL) and saturated aqueous NH₄Cl (5 mL). The organic phase is separated and the aqueous phase is back-extracted with EtOAc (2×30 mL). The combined extracts are dried (MgSO₄), filtered and evaporated. The crude product is purified by preparative TLC developing with MeOH/DCM to afford I-5.

### EXAMPLE 6

### 3-Chloro-5-[6-chloro-2-fluoro-3-(1H-pyrazolo[3,4-c]pyridazin-3-ylmethoxy)-phenoxy]-benzonitrile

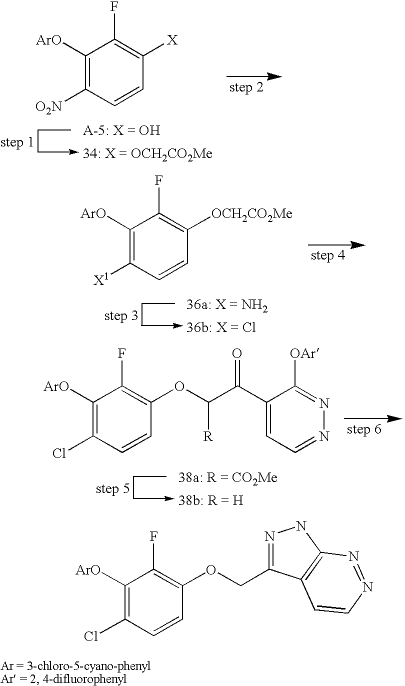

step 1—To a solution of the methyl bromoacetate (4.85 g, 1.5 equiv) and A-5 (6.0 g, 19.4 mmol) in acetone (60 mL) is added anhydrous K₂CO₃ (5.3 g, 2 equiv) and the resulting solution is heated to 60° C. for 2 h. Most of the acetone is removed by evaporation, and the remaining material is partitioned between EtOAc and water. The organic phase is dried (MgSO₄) and the volatile materials are evaporated to afford 34.

step 2—A mixture of 34 (2.28 g, 5.79 mmol), vanadyl acetylacetonate (0.184 g, 0.12 equiv.) and 5% Pd/C (0.525 g, 0.23 WT/equiv.) in THF (23 mL) is stirred under a H₂ atmosphere maintained with a balloon. The suspension is stirred for 36 h then filtered through CELITE®. The solvents are evaporated and the crude product is purified by SiO₂ chromatography eluting with EtOAc/hexanes to afford 36a.

step 3-tert-Butyl nitrite (0.674 mL, 1.3 equiv.) and a solution of 36a (1.60 g, 4.38 mmol) and MeCN (8 mL) are added sequentially to a solution of LiCl (0.371 g, 2 equiv.) and CUCl₂ (0.765 g, 1.3 equiv.) in MeCN (22 mL) heated to 60° C. The reaction mixture is maintained at 60° C. for 2 h then is quenched with 1 N HCl. The aqueous layer is extracted with EtOAc, and the combined organic extracts are dried (MgSO₄), filtered and evaporated. The crude product is purified by SiO₂ chromatography eluting with EtOAc/hexanes to afford 36b.

step 6—To a solution of 30b (605 mg, 2.4 mmol) in DMF (10 mL) is added CDI (410 mg, 2.5 mmol). The mixture is heated to 50° C. under an Ar atmosphere for 1.5 h. The solution is cooled to −10° C. and a solution of 36b (1 g, 2.5 mmol) in DMF (5 mL) is added via syringe. While stirring vigorously, NaH (336 mg, 8.4 mmol) is added in 3 portions over 20 min. The orange solution is stirred for another 10 min and then the cooling bath is removed. The mixture is stirred for 1 h at RT. The reaction mixture is diluted with saturated NH₄Cl solution (20 mL), water (30 mL) and EtOAc (50 mL) then agitated. The EtOAc phase is washed brine (50 mL) and the brine solution is extracted with EtOAc (2×30 mL). The combined extracts are dried (MgSO₄), filtered and evaporated. The crude product is purified by SiO₂ chromatography eluting with an EtOAc/hexane gradient to afford 38a.

step 7—To a solution of 38a (670 mg, 1.06 mmol) in DMSO (8 mL) is added water (0.4 mL) and brine (10 drops). The mixture is heated to 145° C. (oil bath temperature) under Ar atmosphere for 10 min. The solution is cooled to RT and water (60 mL), EtOAc (30 mL) and Et₂O (30 mL) are added. The mixture is agitated and NaCl (2 gm) is added. The mixture is again agitated and the organic phase is collected, washed with brine solution (50%) and the brine solution back-extracted with EtOAc/Et₂O (1:1, 2×50 mL). The combined organic phases are dried (MgSO₄), filtered and evaporated. The crude product is purified by SiO₂ chromatography eluting with EtOAc/hexanes to afford 38b.

step 7—To a solution of 38b (100 mg, 0.17 mmol) in MeOH (2 mL) is added sequentially tert-butyl carbazate (45 mg, 2 eq) and glacial HOAc (0.03 mL). The mixture is heated at 60° C. for 5 h and then is stirred at RT overnight. The mixture is partitioned between DCM (20 mL) and 5% NaHCO₃ (20 mL). The aqueous phase is back-extracted with DCM (2×20 mL) and the combined organic extracts are dried (MgSO₄), filtered and evaporated. This residue is dissolved in THF (4 mL) in a microwave vial, DBU (0.04 mL, 1.5 equivalents) is added and the resulting solution is heated for 10-12 min at 150° C. in microwave. The mixture is partitioned among EtOAc (40 mL), water (30 mL) and saturated aqueous NH₄Cl (5 mL). The organic phase is separated and the aqueous phase is back-extracted with EtOAc (2×30 mL). The combined extracts are dried (MgSO₄), filtered and evaporated. The crude product is purified by preparative SiO₂ chromatography.

### EXAMPLE 7

### 2-Amino-3-methyl-butyric acid 3-[4-bromo-3-(3-chloro-5-cyano-phenoxy)-2-fluoro-benzyl]-pyrazolo[3,4-c]pyridazin-1-ylmethyl ester (40c)

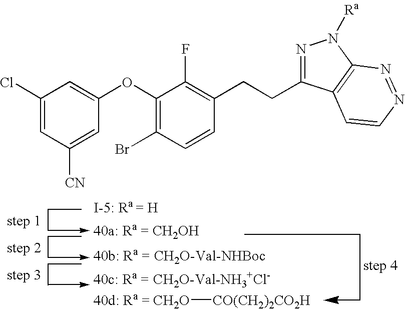

steps 1 and 2—A solution of I-5 (4.3 mmol), MeOH (90 mL) and 37% aqueous CH₂O (18 mL) is heated at reflux. After 1.5 h, the solution is cooled under a stream of nitrogen. The reaction is concentrated and when the volume is reduced to about 30 mL, the solid precipitated and 10g of ice is added. The solid is filtered and stored in vacuo at 50° C. overnight to afford 40a. To a solution of 40a (3.05 mmol), DMF (5 mL) is added sequentially a solution of TEA (0.2 equiv.) and DMF (1 mL) and a solution of N-Boc-valine N-carboxyanhydride (CASRN 141468-55-5, 3.66 mmol) and DMF (2 mL). The resulting solution is stirred at RT for 2.5 h. The mixture is partitioned between water and EtOAc. The aqueous phase is extracted with EtOAc and the combined organic extracts are dried (MgSO₄), filtered and evaporated. The crude product is purified by preparative TLC developed with MeOH/MeOH containing 1% TEA to afford 40b.

step 3—To a mixture of 40b and Et₂O maintained under an N₂ atmosphere is add a solution of HCl in Et₂O (3.5 equiv. HCl, 1 M solution in Et₂O) and the resulting solution is stirred for 4 h at RT. The solid is sedimented in a centrifuge and the solvent decanted. The resulting solid is twice triturated with EtOAc/hexane and the supernatant is discarded. The solid is dried in vacuo to afford 40c.

step 4—The succinate ester is prepared as follows. The hydroxymethyl adduct 40a (3.05 mmol), succinic anhydride (3.2 mmol), DMAP (20 mg, 0.15 mmol), NMM (0.40 mL, 3.7 mmol) are dissolved in DCM (35 mL) and stirred at RT for 2.5 h. The mixture is poured into 0.5 M aqueous KHSO₄ and extracted with DCM. The combined extracts are dried (Na₂SO₄), filtered and evaporated to afford the crude product which is purified by filtration through a pad of SiO₂ eluting with a gradient (2:1 to 3:1 EtOAc/hexane then 3:1 EtOAc/hexane with 0.5% HOAc) to afford 40d.

### EXAMPLE 8

### 3-{6-Bromo-2-fluoro-3-[(1H-pyrazolo[3,4-b]pyridin-3-ylamino)-methyl]-phenoxy}-5-chloro-benzonitrile (I-8)

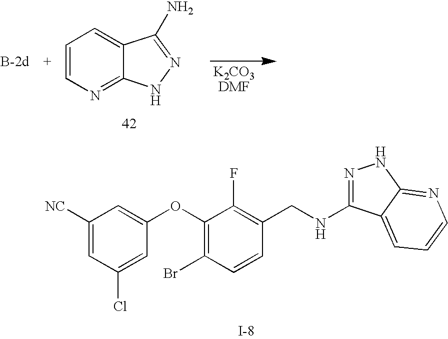

To a solution of B-2d (0.050 g, 0.12 mmol, Ar=3-chloro-5-cyano-phenyl) in DMF (2 mL) was added 42 (CASRN 6752-16-5, 0.019 g, 0.14 mmol) followed by K₂CO₃ (0.020 g, 0.14 mmol). The reaction mixture was heated to 60° C. After 2 h, the reaction mixture was quenched with saturated NH₄Cl, and the aqueous layer was extracted with EtOAc. The combined organic extracts were dried (MgSO₄), filtered and concentrated in vacuo. The product was purified by SiO₂ chromatography eluting with 5% MeOH/DCM to afford 0.010 g (18%) of I-8 as a yellow solid.

3-{6-Bromo-2-fluoro-3-[(1H-pyrazolo[3,4-c]pyridazin-3-ylamino)-methyl]-phenoxy}-5-chloro-benzonitrile is prepared analogously except 42 is replaced with 1H-pyrazolo[3,4-c]pyridazin-3-amine (CASRN 2125-94-2).

I-9 was prepared analogously except B-2d was replaced with 3-(3-bromo-6-bromomethyl-2-fluoro-phenoxy)-5-chloro-benzonitrile which can be prepared from E-2a by sequential reduction with NaBH₀ and conversion of the resulting alcohol to the corresponding bromide with PBr₃ as described in step 4 of example 5.

### EXAMPLE 9

### 3-Chloro-5-[2,6-difluoro-3-(1H-pyrazolo[3,4-c]pyridazin-3-ylmethoxy)-phenoxy]-benzonitrile (I-6)

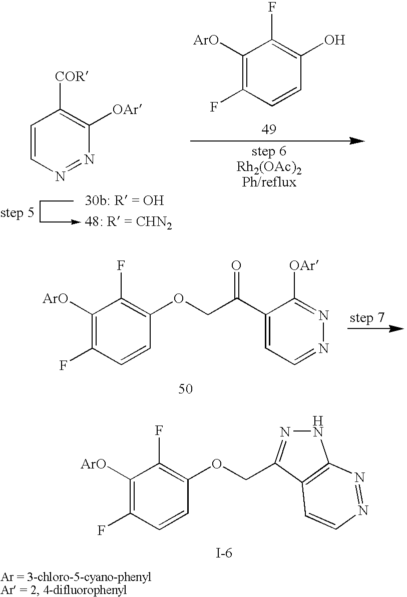

step 5—To an ice-cold solution of 30b (500 mg, 1.98 mmol) and NMM (0.24 μL, 2.2 mmol) in dry THF (20 mL) was added isobutylchloroformate (0.27 μL, 2.1 mmol) dropwise, via syringe. The mixture was stirred for 5 min at 0° C. under a nitrogen atmosphere and then warmed to RT. After 1 h the mixture was filtered through a short plug of CELITE®. To the filtrate was added a 0.3 M solution of ethanol free diazomethane (80 mL, in ether) and the mixture was aged for 30 min. Water (100 mL) was added and the mixture was transferred to a separatory funnel. The organic phase was isolated and the aqueous phase back extracted with ether (80 μL). The combined ether phases were dried (MgSO₄), filtered and concentrated. The crude product was purified by SiO₂ chromatography eluting with a EtOAc/hexane gradient (30 to 50% EtOAc) to afford 0.250 g of 48 as a red-orange solid.

step 6—A solution of 49 (102 mg, 0.36 mmol) and rhodium(II) acetate dimer (8 mg, 0.02 mmol) in dry benzene (3.5 mL) was heated to 80° C. under nitrogen atmosphere. To this mixture was added a solution of 48c (50 mg, 0.18 mmol) in dry benzene (2 mL), over 40 min, via syringe pump. After the addition was completed, the mixture was stirred for 20 min. The mixture was cooled to RT and water (30 mL) and EtOAc (30 mL) were added. The EtOAc phase was separated and the aqueous phase back-extracted with EtOAc(2×30 mL). The combined extracts were dried (MgSO₄), filtered and concentrated. The residue was purified by SiO₂ chromatography on a preparative TLC plate developed with 42% EtOAc/hexanes to afford 17 mg of semi-pure 50 as a light yellow viscous oil.

step 7—To a solution of 50 (57 mg, 07 mmol, 65% pure) and pTsOH monohydrate (44 mg, 0.23 mmol) in IPA (4 mL) was added hydrazine hydrate (8 mg, 0.14 mmol). The mixture was heated to 80° C. for 9 h. An 20% aqueous solution of Na₂CO₃ (1 mL) and water (2 mL) was added and the mixture was stirred for 5 min. The solution was partitioned between 20% Na₂CO₃ (2 mL), water (30 mL) and EtOAc (30 mL). The aqueous phase was back-extracted with EtOAc (2×30 mL) and combined EtOAc phases, dried (MgSO₄), filtered and concentrated. The residue was purified by preparative SiO₂ plate developed with 70% EtOAc/hexanes) followed by a second plate developed with 7% MeOH/DCM to afford 0.005 g of I-6 as a white solid.

### EXAMPLE 10

### 3-[3-Bromo-2-fluoro-6-(1H-pyrazolo[3,4-c]pyridazin-3-ylmethoxy)-phenoxy]-5-chloro-benzonitrile (I-7)

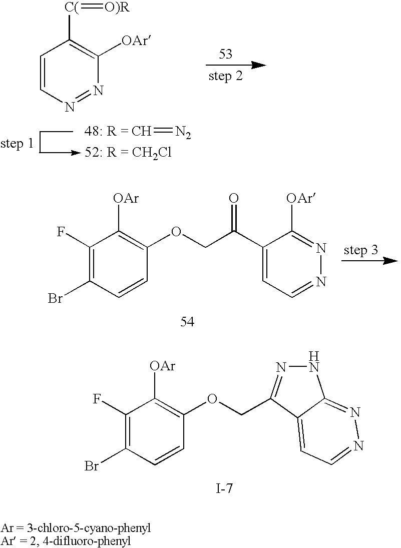

step 1—A solution of 48 (1 g, 3.6 mmol) and dioxane (2.5 mL) is gently warmed in a water bath to solubilize the material. When the solution is homogeneous the solution is cooled to RT, diluted with Et₂O (15 mL) and then a 10% aqueous HCl solution (3.5 mL) was added. The mixture was stirred vigorously for 40 min. Et₂O (40 mL) is added and the mixture is basified with 5% aqueous NaHCO₃. Water (60 mL) was added and the mixture was transferred to a separatory funnel. The organic phase was isolated and washed with brine (60 mL). The aqueous phase was back extracted with ether (60 mL). The combined ether phases were dried (MgSO₄), filtered and concentrated to provide 52 as a orange-brown semi-viscous oil which was used immediately in the next step.

step 2—A solution of 3-(3-bromo-2-fluoro-6-hydroxy-phenoxy)-5-chloro-benzonitrile (53, 45 mg, 0.14 mmol), K₂CO₃ (42 mg, 0.3 mmol) and 52 (40 mg, 0.14 mmol) in DCE (2.5 mL) in a sealed microwave tube was heated to 100° C. for 30 min. An additional amount of 52(45 mg) and K₂CO₃ (42 mg) was added and the mixture was heated to 120° C. for an additional 30 min. Potassium iodide was added and the mixture heated to 120° C. for 30 minutes and then 140° C. for 1 hour. The solution was cooled to RT and partitioned between H₂O (20 mL) and EtOAc (20 mL). The EtOAc solution was washed with brine (20 mL). The aqueous phase was back-extracted with EtOAc (2×20 mL) and the combined organic extracts dried (MgSO₄), filtered and concentrated. The crude product was purified by preparative SiO₂ chromatography and developed with 47% EtOAc/hexanes) to afford 0.033 g of 54 as a red oil.

step 3—To a solution of 54 (33 mg, 14 mmol) and pTsOH monohydrate (22 mg, 0.12 mmol) in IPA (1.5 mL) was added hydrazine hydrate (8 mg, 0.14 mmol). The mixture was heated to 80° C. for 8 h, cooled and aqueous 20% Na₂CO₃ (1 mL) and water (2 mL) were added and the mixture stirred for 5 minutes. A 20% Na₂CO₃ solution (2 mL), water (30 mL) and EtOAc (30 mL) were added. The phases were separated and the water was extracted with EtOAc (2×30 mL), combine EtOAc phases, dried (MgSO4), filtered and evaporated. The crude product was purified by preparative SiO₂ chromatography and developed with 70% EtOAc/hexanes) to afford 2 mg of I-7 as an off-white solid.

### EXAMPLE 11

### HIV-1 Reverse Transcriptase Assay

RNA-dependent DNA polymerase activity was measured using a biotinylated primer oligonucleotide and tritiated dNTP substrate. Newly synthesized DNA was quantified by capturing the biotinylated primer molecules on streptavidin coated Scintillation Proximity Assay (SPA) beads (Amersham). The sequences of the polymerase assay substrate were: 18nt DNA primer, 5′-Biotin/GTC CCT GTT CGG GCG CCA-3′; 47nt RNA template, 5′-GGG UCU CUC UGG UUA GAC CAC UCU AGC AGU GGC GCC CGA ACA GGG AC-3′. The biotinylated DNA primer was obtained from the Integrated DNA Technologies Inc. and the RNA template was synthesized by Dharmacon. The DNA polymerase assay (final volume 50 μl) contained 32 nM biotinylated DNA primer, 64 nM RNA substrate, dGTP, dCTP, dTTP (each at 5 μM), 103 nM [³H]-dATP (specific activity=29 μCi/mmol), in 45 mM Tris-HCl, pH 8.0, 45 mM NaCl, 2.7 mM Mg(CH₃COO)₂, 0.045% Triton X-100 w/v, 0.9 mM EDTA. The reactions contained 5 ul of serial compound dilutions in 100% DMSO for IC50 determination and the final concentrations of DMSO were 10%. Reactions were initiated by the addition of 30 μl of the HIV-RT enzyme (final concentrations of 1-3 nM). Protein concentrations were adjusted to provide linear product formation for at least 30 min of incubation. After incubation at 30° C. for 30 min, the reaction was quenched by addition of 50 μl of 200 mM EDTA (pH 8.0) and 2 mg/ml SA-PVT SPA beads (Amersham, RPNQ0009, reconstituted in 20 mM Tris-HCl, pH 8.0, 100 mM EDTA and 1% BSA). The beads were left to settle overnight and the SPA signals were counted in a 96-well top counter-NXT (Packard). IC₅₀ values were obtained by sigmoid regression analysis using GraphPad. Representative values are tabulated in TABLE II.

TABLE II

| Compound | IC₅₀ (μM) |
|---|---|
| I-2 | 0.0244 |
| I-3 | 0.011 |

### EXAMPLE 12

### Antiviral Assay Method

Anti-HIV antiviral activity was assessed using an adaptation of the method of Pawls et al. (J. Virol Methods 1988 20:309-321). The method is based on the ability of compounds to protect HIV-infected T lymphoblastoid cells (MT4 cells) from cell-death mediated by the infection. The endpoint of the assay was calculated as the concentration of compound at which the cell viability of the culture was preserved by 50% (‘50% inhibitory concentration’, IC₅₀). The cell viability of a culture was determined by the uptake of soluble, yellow 3-[4,5-dimethylthiazol-2-yl]-2,5-diphenyltetrazolium bromide (MTT) and its reduction to a purple insoluble formazan salt. After solubilization, spectrophotometric methods were employed to measure the amount of formazan product.

MT4 cells were prepared to be in logarithmic-phase growth and a total of 2×10⁶ cells infected with the HXB2-strain of HIV at a multiplicity of 0.0001 infectious units of virus per cell in a total volume of between 200-500 microliters. The cells were incubated with virus for one hour at 37° C. before removal of virus. The cells are then washed in 0.01 M phosphate buffered saline, pH 7.2 before being resuspended in culture medium for incubation in culture with serial dilutions of test compound. The culture medium used was RPMI 1640 without phenol red, supplemented with penicillin, streptomycin, L-glutamine and 10% fetal calf serum (GM10).

Test compounds were prepared as 2 mM solutions in dimethyl sulphoxide (DMSO). Four replicate, serial 2-fold dilutions in GM10 were then prepared and 50 microliters amounts placed in 96-well plates over a final nanomolar concentration range of 625-1.22. Fifty microliters GM10 and 3.5×10⁴ infected cells were then added to each well. Control cultures containing no cells (blank), uninfected cells (100% viability; 4 replicates) and infected cells without compound (total virus-mediated cell death; 4 replicates) were also prepared. The cultures were then incubated at 37° C. in a humidified atmosphere of 5% CO₂ in air for 5 days.

A fresh solution of 5 mg/mL MTT was prepared in 0.01 M phosphate buffered saline, pH 7.2 and 20 microliters added to each culture. The cultures were further incubated as before for 2 hours. They were then mixed by pipetting up and down and 170 microliters of Triton X-100 in acidified isopropanol (10% v/v Triton X-100 in 1:250 mixture of concentrated HCl in isopropanol). When the formazan deposit was fully solubilized by further mixing, the absorbance (OD) of the cultures was measured at 540 nm and 690 nm wavelength (690 nm readings were used as blanks for artifacts between wells). The percent protection for each treated culture was then calculated from the equation:

$$\% Protection = \frac{\begin{matrix}( OD drug treated cultures ) - \\ ( OD untreated virus control cultures )\end{matrix}}{\begin{matrix}( OD uninfected cultures ) - \\ ( OD untreated virus control cultures )\end{matrix}} \times 100 \%$$

The IC₅₀ can be obtained from graph plots of percent protection versus log₁₀ drug concentration. Representative values are tabulated in TABLE III.

TABLE III

| | Antiviral Assay |
|---|---|
| Compound | IC50 (νM) |
| I-2 | 0.0037 |
| I-3 | 0.008 |

### EXAMPLE 13

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |

| | |
|---|---|
| Distilled water | q.s. to 100 ml |

The ingredients are mixed to form a suspension for oral administration.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A compound according to formula (I) 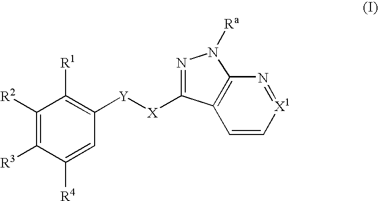
wherein:
X is CH₂ or NH;
Y is CH₂ or O with the proviso that at least one of X or Y is CH₂; and with the further proviso that when X¹ is CH, either (i) R¹ is OAr or C(═O)Ar or (ii) X is NH
X¹ is N or CH;
R¹ is C(═O)Ar, OAr, fluorine or hydrogen;
R² is OAr, hydrogen, halogen, C₁₋₆ alkyl, C₁₋₆ alkoxy or C₃₋₅ cycloalkyl;
R³ and R⁴ are independently hydrogen, halogen, C₁₋₆ alkyl, C₁₋₆ alkoxy or C₃₋₅ cycloalkyl;
R^{a} is hydrogen, CH₂OH, CH₂C(═O)(CH₂)ₙC(═O)OH where n is 2 to 5, CH₂C(═O)C₁₋₆ alkyl, or CH₂C(═O)CHR^{b}NH₂ where R^{b} is phenyl or C₁₋₆ lower alkyl;
Ar is phenyl substituted with 1 to 3 groups independently selected from halogen, cyano, C₁₋₆ haloalkyl or C₁₋₆ alkyl; or,
pharmaceutically acceptable salts thereof.

2. A compound according to claim 1 wherein R¹ is hydrogen or fluorine and R² is OAr.

3. A compound according to claim 2 wherein:
R¹ is fluoro;
R³ is halogen, C₁₋₆ alkyl, C₁₋₆ alkoxy or C₃₋₅ cycloalkyl; and,
R⁴ and R^{a} are hydrogen.

4. A compound according to claim 3 wherein
Ar is a moiety of formula (i) 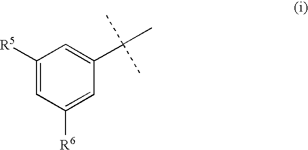
R⁵ is cyano; and,
R⁶ is halogen, cyano or C₁₋₆ haloalkyl.

5. A compound according to claim 4 wherein X¹ is N, X is CH₂ and Y is CH₂ or O.

6. A compound according to claim 5 wherein Y is O.

7. A compound according to claim 5 wherein Y is CH₂.

8. A compound according to claim 4 wherein Y is CH₂ and X is NH.

9. A compound according to claim 4 wherein X¹ is CH and X is NH.

10. A compound according to claim 2 wherein
X¹ is N;
X is CH₂;
Y is CH₂ or O;
R¹ is fluoro;
R³ is halogen, C₁₋₆ alkyl, C₁₋₆ alkoxy or C₃₋₅ cycloalkyl; and,
R⁴ is hydrogen;
Ar is a moiety of formula (I) 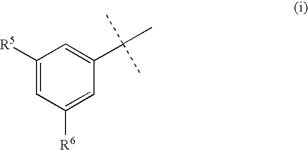
R⁵ is cyano;
R⁶ is halogen, cyano or C₁₋₆ haloalkyl; and,
R^{a} is CH₂C(═O)(CH₂)ₙC(═O)OH where n is 2 to 5.

11. A compound according to claim 2 wherein:
R¹ and R⁴ are fluoro;
R³ is halogen, C₁₆ alkyl, C₁₋₆ alkoxy or C₃₋₅ cycloalkyl; and,
R^{a} is hydrogen or CH₂C(═O)(CH₂)ₙC(═O)OH where n is 2 to 5.

12. A compound according to claim 11 wherein:
X¹ is N;
X is CH₂;
Y is CH₂ or O;
Ar is a moiety of formula (i) 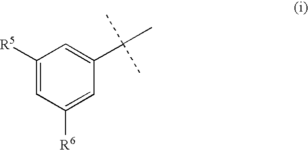
R⁵ is cyano; and,
R⁶ is halogen, cyano or C₁₋₆ haloalkyl.

13. A compound according to claim 12 wherein R^{a} is hydrogen.

14. A compound according to claim 1 wherein said compound is of formula I wherein R¹ is OAr and R², R³ and R⁴ are independently hydrogen, halogen or C₁₋₆ alkyl.

15. A compound according to claim 14 wherein:
R⁴ is hydrogen;
R^{a} is CH₂C(═O)(CH₂)ₙC(═O)OH wherein n is 2 to 5 or hydrogen;
Ar is a moiety of formula (i) 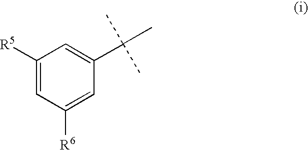
R⁵ is cyano; and R⁶ is halogen, cyano or C₁₋₆ haloalkyl

16. A compound according to claim 15 wherein R^{a} is hydrogen.

17. A compound according to claim 15 wherein X¹ is N.

18. A compound according to claim 15 wherein X¹ is CH.

19. A compound according to claim 1 wherein R¹ is C(═O)Ar and R² and R³ are independently hydrogen, halogen or C₁₋₆ alkyl.

20. A compound according to claim 19 wherein:
R^{a} is hydrogen;
Ar is a moiety of formula (i) 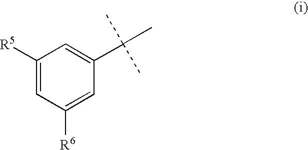
R⁵ is cyano; and,
R⁶ is halogen, cyano or C₁₋₆ haloalkyl.

21. A compound according to claim 20 wherein R² is halogen and R³ is halogen or C₁₋₆ alkyl.

22. A compound according to claim 21 wherein X¹ is N.

23. A compound according to claim 21 wherein X¹ is CH.

24. A method for treating an HIV-1 infection, or preventing an HIV-1 infection, or treating AIDS or ARC, comprising administering to a host in need thereof a therapeutically effective amount of a compound according to claim 1.

25. A method for treating HIV-1 infection according to claim 24 further comprising co-administering at least one compound selected from the group consisting of HIV protease inhibitors, nucleoside reverse transcriptase inhibitors, non-nucleoside reverse transcriptase inhibitors, integrase inhibitors, CCR5 antagonists and viral fusion inhibitors.

26. A method according to claim 25 wherein said nucleoside reverse transcriptase inhibitor is zidovudine, lamivudine, didanosine, zalcitabine, stavudine, rescriptor, sustiva or viramune; said non-nucleoside reverse transcriptase inhibitor is efavirenz, nevirapine, delavirdine or etravirine, and/or said protease inhibitor is saquinavir, ritonavir, nelfmavir, indinavir, amprenavir, or lopinavir and/or the viral fusion inhibitor is enfuvirtide and/or said CCR5 antagonist is maraviroc and/or said integrase inhibitor is raltegravir.

27. A method for inhibiting HIV reverse transcriptase in a host infected with HIV-1 comprising administering a therapeutically effective amount of a compound according to claim 1.

28. A method according to claim 27 wherein the host is infected with a strain of HIV-1 expressing a reverse transcriptase with at least one mutation compared to wild type HIV.

29. A method according to claim 28 wherein said reverse transcriptase exhibits reduced susceptibility to efavirenz, nevirapine or delavirdine.

30. A pharmaceutical composition for treating an HIV-1 infection, or preventing an HIV-1 infection, or treating AIDS or ARC, comprising a therapeutically effective quantity of a compound according to claim 1 and at least one carrier, excipient or diluent.
